(19) United States
(12) Patent Application Publication
(10) Pub. No.: US 2023/0008576 A1
(43) Pub. Date: Jan. 12, 2023

(54) A HOST-MICROBE CO-CULTURE PERFUSION BIOREACTOR FOR DISCOVERY OF SECRETED PRODUCTS AND NOVEL INTERACTIONS AT THE HUMAN-MICROBIOTA INTERFACE

(71) Applicant: Merck Sharp & Dohme LLC, Rahway, NJ (US)

(72) Inventors: Stephen H. Kasper, Watertown, MA (US); Erik C. Hett, Arlington, MA (US); Jason Cassaday, Coopersburg, PA (US); Kumael Jafri, Holliston, MA (US); Thomas Philip Wyche, Chelsea, MA (US)

(73) Assignee: Merck Sharp & Dohme LLC, Rahway, NJ (US)

(21) Appl. No.: 17/779,013

(22) PCT Filed: Nov. 20, 2020
(86) PCT No.: PCT/US2020/061440

§ 371 Date: May 23, 2022

Related U.S. Application Data
(60) Provisional application No. 62/940,610, filed on Nov. 26, 2019.

(51) Int. Cl. C12M 1/00; C12M 1/04

(52) U.S. Cl. CPC C12M 29/10; C12M 23/34; C12M 29/04; C12M 1/045

(57) ABSTRACT

The present invention relates to a perfusion bioreactor for co-culturing, wherein the bioreactor grows cells in two separate environments and enables communication across environments and populations. The chambers' contents are continuously mixed to expose the environment (or cell population) of each chamber to the secreted products of the other chamber. The said bioreactor comprises at least, but not limited to, two chambers, with separate cell populations with at least two separate environments independently selected from aerobic or anaerobic environment and media favorable to cell growth. The bioreactor allows for multiple samples to be collected during an experiment to enable various analytical techniques and results. Additionally, the bioreactor comprises a multi-chamber cell culture system capable of emulating the gastro-intestinal tract.

### BACKGROUND OF THE INVENTION

The human microbiome refers to the communities of microorganisms living in association with our bodies. In recent years, the human microbiome has become recognized as a master regulator of health and disease, and therefore, presents an opportunity for uncovering novel interactions between microbes and the human host. The gastrointestinal tract microbiome is known to contain bacteria, fungi, bacteriophages and other viruses, and is known to contribute to a variety of human diseases.

In order to achieve optimal results in cell and tissue culture, bioreactors should ideally operate under conditions that are as close as possible to in vivo conditions. Difficulties have arisen with known bioreactors in that they have not provided a constant and regular supply of nutrition and removal of metabolic byproducts.

In vitro models typically grow bacterial cells or human cells independently, in isolation of other types of cells.

The bacteria that comprise the human microbiome encode a wealth of secreted products. In order to gain a functional understanding of these products, it is necessary to grow bacterial and human cells in proximity to one another. Common laboratory culture conditions that are conducive to one cell type (e.g., human cell) are often deleterious to the other cell type (e.g., bacteria). Additionally, direct contact of bacteria and eukaryotic cells provides the opportunity for cellular infection and viability loss, often making the time scale of co-culture finite.

Previous attempts at mimicking the human gut-microbiota interactions include Gut-on-Chip (Kim et al, Lab Chip (2012)) and HuMix (Shah et al., Nat Comm (2016)). However, these two applications, while useful for testing compounds or reductionist biological hypotheses in controlled systems including human-cell based, are not optimized for bioreactor-like applications and only allow for a small volume and subsequently, limited analysis.

Cells are known to have secreted signals. In one example, bacteria may utilize biosynthetic gene cluster to synthesize secreted molecules with human GPCR, or other surface receptor, ligand activity (e.g., commendamide, Nature 549, 48-53 (2017)). In another example, human cells may secrete antimicrobial peptides and galectins to modulate bacterial growth (Immunity 42(1), 28-39 (2015)). The bacteria in turn can secrete proteases, which may degrade host defense peptides (J. Biol. Chem. 271(17). 10079-86 (1996)), or outer membrane vesicles, which may sequester peptides (J. Prot. Res. 13(3), 1345-58 (2014)). Outer membrane vesicles often carry multiple contents that can affect multiple processes. In another example, bacteria may secrete siderophores to acquire and/or quench extracellular metal ions; whereas human cells may secrete iron binding proteins (e.g., lactoferrin). This scavenging competition of metal ions may then affect cellular enzymatic processes (e.g., DNA replication, ATP synthesis) that require metal co-factors (Cell. Microbiol. 12(12), 1691-1702 (2010)). In another example, bacteria may alter the reduction-oxidation (redox) potential environment by secreting redox active molecules (e.g., hydrogen sulfide, nitric oxide, pyocyanin, indole, uracil), thereby altering a variety of host cell effects (Free Radic. Biol. Med. 105, 110-131 (2017)). In another example, bacteria may secrete host protease inhibitors that modulate secreted protein activity (Cell 168(3), 517-526 (2017)). In another example, bacteria may secrete proteases that modulate the membrane associated proteins of human cells, thereby modulating cellular morphology and inducing changes in cellular signaling programs (EMBO Rep. 11, 798-804 (2010); Proc. Natl. Acad. Sci. USA 95, 14979-14984 (1998)). In another example, bacteria may secrete membrane-disruptive toxins, thereby modulating cellular morphology and inducing changes in cellular signaling programs and/or release of intracellular components into the extracellular environment (Zentralbl. Bakteriol. 284(2-3), 170-206 (1996)).

### SUMMARY OF THE INVENTION

The present invention relates to a perfusion bioreactor for co-culturing cells, wherein the bioreactor grows cells in two separate environments and enables communication across cells grown in different chambers, environments, or populations. When applicable, the contents inside or outside the chambers' membranes are continuously mixed with the contents inside or outside of the membrane in a different chamber to expose the environment (or cell population) to the secreted products of the other chamber. When applicable, the contents inside or outside the chamber's membrane is mixed with the contents of the separate environment to expose the environment (or cell population) to the secreted products of the other chamber, wherein the separate chamber has no membrane.

The present invention relates to a perfusion bioreactor comprising: (a) a first chamber, wherein the first chamber comprises an inner membrane defining an inner volume, and an outer wall surrounding the inner membrane and defining an outer volume surrounding the inner membrane; (b) a second chamber, wherein the second chamber comprises an optional inner membrane defining an inner volume, and an outer wall surrounding the optional inner membrane, wherein if the inner membrane is present, then the outer wall defines an outer volume surrounding the inner membrane, and if the inner membrane is not present, then the outer wall defines an outer volume comprising all the contents in the chamber, (c) a hollow conduit connecting the first and second chambers via an orifice in each of the first and second chambers, wherein the hollow conduit allows fluid communication between the first and second chamber; and (d) a first pump in mechanical contact with or in fluid communication with the hollow conduit, the first pump to move fluid between the first chamber and the second chamber.

The present invention relates to a perfusion bioreactor comprising: (a) a first chamber, wherein the first chamber comprises an inner membrane defining an inner volume, and an outer wall surrounding the inner membrane and defining an outer volume surrounding the inner membrane; (b) a second chamber, wherein the second chamber comprises an inner membrane defining an inner volume, and an outer wall surrounding the inner membrane, and defining an outer volume surrounding the inner membrane, (c) a hollow conduit connecting the first and second chambers via an orifice in each of the first and second chambers, wherein the hollow conduit allows fluid communication between the first and second chamber; and (d) a first pump in mechanical contact with or in fluid communication with the hollow conduit, the first pump to move fluid between the first chamber and the second chamber.

The present invention relates to a perfusion bioreactor comprising: (a) a first chamber, wherein the first chamber comprises an inner membrane defining an inner volume, and an outer wall surrounding the inner membrane and defining an outer volume surrounding the inner membrane; (b) a second chamber, wherein the second chamber does not comprise an inner membrane, and an outer wall surrounding the chamber, and wherein the outer wall defines an outer volume comprising all the contents in the chamber, (c) a hollow conduit connecting the first and second chambers via an orifice in each of the first and second chambers, wherein the hollow conduit allows fluid communication between the first and second chamber; and (d) a first pump in mechanical contact with or in fluid communication with the hollow conduit, the first pump to move fluid between the first chamber and the second chamber.

The present invention relates to a bioreactor comprising at least, but not limited to, two-chambers, wherein the bioreactor grows cells in two separate environments and enables communication between cells grown in different chambers. The present invention relates to co-culturing at least two separate cell populations (e.g., human cells, microbial cells, or any combination thereof).

The bioreactor comprises at least, but not limited to, two chambers, with separate cell populations with at least two separate environments independently selected from an aerobic or an anaerobic environment. The aerobic environment may comprise epithelial cells or microbial cells. The anaerobic environment may comprise bacterial cells.

In an embodiment, the bioreactor system is a bioreactor. In an embodiment, the bioreactor system comprises chambers, wherein each chamber represents a bioreactor.

In particular, the present invention comprises a bioreactor for co-culturing two separate cell types (or populations) with two separate environments, such as i) aerobic and aerobic conditions, ii) aerobic and anaerobic conditions, or iii) anaerobic and anaerobic conditions.

The present invention comprises a bioreactor for co-culturing at least two separate cell populations with at least two separate environments independently selected from i) mammalian cells in aerobic conditions, ii) microbial cells in aerobic conditions, iii) microbial cells in anaerobic conditions. The present invention comprises a bioreactor for co-culturing at least two separate cell populations with at least two separate environments independently selected from i) human cells in aerobic conditions, ii) microbial cells in aerobic conditions, iii) microbial cells in anaerobic conditions. The present invention comprises a bioreactor for co-culturing at least two separate cell populations with at least two separate environments independently selected from i) epithelial cells in aerobic conditions, ii) microbial cells in aerobic conditions, iii) microbial cells in anaerobic conditions.

An embodiment of the present invention comprises human epithelial cells. An embodiment of the present invention comprises mammalian cells. An embodiment of the present invention comprises human endothelial cells. In one embodiment of the present invention, microbial cells comprise bacteria cells.

In an embodiment of the invention, the bioreactor comprises two chambers. In a further embodiment, the bioreactor comprises at least two chambers, wherein one chamber comprises microbial cells and the a different chamber comprises human cells. In a further embodiment, the human cells are human epithelial cells. In another embodiment, the human epithelial cells grow on a porous, matrix-coated (e.g., collagen) membrane scaffold. In yet a further embodiment, the microbial chamber harnesses bacteria within a membrane maintained in an anaerobic or aerobic environment. In another embodiment, the microbial chamber comprises a mucin-coated inner membrane. In yet another embodiment, said chamber comprising human cells is maintained under aerobic conditions. In an embodiment, the chambers comprise a compartment to introduce other cell types (such as immune cells).

The present invention relates to a multi-chamber system, representing a bioreactor, comprising at least two chambers, wherein each chamber houses a separate cell population. In a further embodiment, the contents from both chambers are mixed and each chamber is exposed to the contents of the other chamber(s). In a further embodiment, the contents are continuously mixed.

In an embodiment, the bioreactor system operates with or without an inner membrane in each chamber. In a further embodiment, the bioreactor system comprises chambers, wherein all chambers within the bioreactor system comprise an inner membrane. In another embodiment, the bioreactor system comprises at least one chamber with an inner membrane and at least one chamber without an inner membrane.

In one embodiment, a membrane can be coated for functionalization. In another embodiment, the membrane is optionally coated to further promote cell adhesion and/or growth. In another embodiment, the membrane comprises a semi-permeable membrane. In a further embodiment, the membrane is an artificial semi-permeable membrane, wherein the membrane facilitates the flow of molecules in solution based on differential diffusion. In one embodiment, the membrane is a dialysis tube. In another embodiment, the cells are located on the inside of the membrane in the chamber. In another embodiment, the cells are located inside a dialysis tube.

In yet a further embodiment, a membrane can be coated for optimal monolayer formation by a Caco-2 epithelial cell layer. A method has been developed for coating dialysis bags with collagen for optimal monolayer formation. Applicable membranes for cell types are those known in the art and include, but are not limited to, the Repligen Biotech cellulose ester dialysis membranes.

The present invention relates to a multi-chamber system, representing a bioreactor, comprising at least two chambers, wherein each chamber comprises a membrane.

In one embodiment, the membrane can be located as a compartment within a chamber and divide the chamber into sections, wherein one section is inside the membrane and one section is outside the membrane. In another embodiment, the membrane can bisect the chamber. In a further embodiment, the membrane is a tube within the chamber.

In one embodiment, the contents inside the membrane of one chamber is the inner volume. In one embodiment, the chamber comprises an outer wall surrounding the inner membrane and defining an outer volume surrounding the inner membrane. In a further embodiment, the contents outside the membrane of one chamber is the outer volume.

In one embodiment, the contents inside the membrane of one chamber are mixed with the contents outside the membrane of a separate chamber. In one embodiment, the contents inside the membrane of one chamber are mixed with the contents inside the membrane of a separate chamber.

In one embodiment, the contents from the outside of the membrane in one chamber are mixed with the contents inside the membrane of a separate chamber. In one embodiment, the contents from the outside of the membrane in one chamber are mixed with the contents outside the membrane of a separate chamber.

In another embodiment, the contents from the inside of the membrane in one chamber are mixed with the contents inside the membrane in a second chamber, and the contents from the outside of the membrane in the second chamber are mixed with the contents outside the membrane of the first chamber.

In another embodiment, the contents from the inside of the membrane in one chamber are mixed with the contents inside the membrane in a second chamber, and the contents from the outside of the membrane in the second chamber are mixed with the contents inside the membrane of the first chamber.

In another embodiment, the contents from the inside of the membrane in one chamber are mixed with the contents outside the membrane in a second chamber, and the contents from the outside of the membrane in the second chamber are mixed with the contents outside the membrane of the first chamber.

In another embodiment, the contents from the inside of the membrane in one chamber are mixed with the contents outside the membrane in a second chamber, and the contents from the outside of the membrane in the second chamber are mixed with the contents inside the membrane of the first chamber.

In another embodiment, the contents from the inside of the membrane in one chamber are mixed with the contents inside the membrane in a second chamber, and the contents from the inside of the membrane in the second chamber are mixed with the contents inside the membrane of the first chamber

In another embodiment, the contents from the inside of the membrane in one chamber are mixed with the contents inside the membrane in a second chamber, and the contents from the inside of the membrane in the second chamber are mixed with the contents outside the membrane of the first chamber

In another embodiment, the contents from the inside of the membrane in one chamber are mixed with the contents outside the membrane in a second chamber, and the contents from the inside of the membrane in the second chamber are mixed with the contents outside the membrane of the first chamber

In another embodiment, the contents from the outside of the membrane in one chamber are mixed with the contents outside the membrane in a second chamber, and the contents from the outside of the membrane in the second chamber are mixed with the contents outside the membrane of the first chamber

In another embodiment, the contents from the outside of the membrane in one chamber are mixed with the contents outside the membrane in a second chamber, and the contents from the outside of the membrane in the second chamber are mixed with the contents inside the membrane of the first chamber

In another embodiment, the contents from the outside of the membrane in one chamber are mixed with the contents inside the membrane in a second chamber, and the contents from the outside of the membrane in the second chamber are mixed with the contents inside the membrane of the first chamber

In another embodiment, the present invention relates to a multi-chamber system, representing a bioreactor, comprising at least two chambers, wherein one chamber does not comprise a membrane.

In one embodiment, the contents inside of the membrane in one chamber is mixed with the contents inside the second chamber, wherein the second chamber does not comprise a membrane. In one embodiment, the contents outside of the membrane in one chamber is mixed with the contents inside the second chamber, wherein the second chamber does not comprise a membrane.

In one aspect of the invention, the modular design of the bioreactor system comprises variation in orientations for different applications. In one embodiment, the bioreactor is modular with at least two chambers. In a further embodiment, additional chambers can be added to the bioreactor setup. In a further embodiment, the continuous mixing of the chamber contents enable communication across cells grown in separate environments. In a further embodiment, the modularity of the bioreactor setup allows for different types of co-culture experiments.

In one aspect of the invention, the bioreactor comprises two chambers, wherein the chambers each house a separate cell type. In one aspect of the invention, the bioreactor comprises two chambers, wherein one chamber houses a microbial culture and the second chamber houses an epithelial culture, wherein the microbial culture comprising microbial cell media is supplied to the microbial chamber inside or outside the dialysis tube, and wherein the epithelial culture comprising epithelial cell media is supplied to the basal side of cells in the epithelial chamber, outside the dialysis tube. In one aspect of the invention, the bioreactor comprises two chambers, wherein one chamber houses a microbial culture and the second chamber houses an human cell culture, wherein the microbial culture comprising microbial cell media is supplied to the microbial chamber inside or outside the dialysis tube, and wherein the human cell culture comprising human cell media is supplied to the basal side of cells in the human cell chamber, outside the dialysis tube. In a further embodiment of the invention, the human cell media applied to the basal side of the cells in the epithelial cells, outside the dialysis tube, mimics the vascular system of the human body.

In an embodiment of the invention, the secreted microbial products outside the dialysis tube in the microbial chamber and on the apical side of cells insider the dialysis tube in the epithelial chamber are mixed.

In an embodiment of the invention, the secreted microbial products outside the dialysis tube in the microbial chamber and on the apical side of cells insider the dialysis tube in the epithelial chamber are continuously mixed.

In one aspect of the present invention, upon co-culture (exposing each chamber to the secreted products of the other chamber(s), the cells can sense and respond to these secreted signals, continuously shaping the secreted cell-cell dialogue. In another embodiment, the multi-chamber cell culture bioreactor emulates the gastrointestinal tract, wherein the chambers comprise separate environments of cells, and additionally, wherein the cell culture contents mix to expose the cell culture of the separate environments to the secreted products of the other environments. In a further embodiment, said mixing exposes the cell culture to the secreted products of the other chambers enabling communication (or crosstalk) across the cells grown in specific environments.

In another embodiment, the multi-chamber cell culture bioreactor emulates the gastrointestinal tract, wherein the chambers comprise separate environments of cells, and additionally, wherein the cell culture contents continuously mix to expose the cell culture of the separate environments to the secreted products of the other environments. In a further embodiment, said continuous mixing exposes the cell culture to the secreted products of the other chambers enabling communication (or crosstalk) across the cells grown in specific environments. In one aspect of the present invention, the cells can sense and respond to secreted signals upon co-culture, continuously shaping the secreted dialogue. In another embodiment, the growth of the environments is directly impacted by the exposure of the cell population and secreted products of the other chamber(s). In one embodiment, mammalian cells respond to the bacteria present through bioluminescence induction. In one aspect of the present invention, the present invention comprises a multi-chamber bioreactor for growing cells, wherein the bioreactor comprises:

at least two separate chambers, wherein each chamber comprises cells;

wherein the cells in each chamber are cultured in media and under environmental conditions favorable to growth;

wherein at least one chamber comprises a membrane, and wherein the membrane divides the chamber into two sections, wherein the two sections comprise the contents inside the membrane and the contents outside the membrane; and

wherein the contents from the chambers mix, thereby exposing the cells in each chamber to the secreted products from a separate chamber(s).

In a further embodiment, the contents from the chambers continuously mix.

In one aspect of the present invention, the present invention comprises the use of a dual chamber bioreactor for growing cells, wherein the bioreactor comprises:

two separate chambers wherein each chamber comprises cells;

wherein the cells in each chamber are cultured in media and under environmental conditions favorable to growth;

wherein each chamber comprises a membrane, and wherein the membrane divides the chamber into two sections, wherein the two sections comprise the contents inside the membrane and the contents outside the membrane; and

wherein the contents from both chambers mix, thereby exposing the cells in each chamber to the secreted products from a separate chamber.

In a further embodiment, the contents from the chambers continuously mix.

In one embodiment, the bioreactor allows for multiple samples to be collected throughout an experiment, cell culture, or harvest. In a further embodiment, the multiple samples enable various analytical techniques and results. In a further embodiment, multiple samples comprise one or more samples taken from the bioreactor throughout the duration of an experiment, cell culture, or harvest. In a further embodiment, the bioreactor allows for multiple samples to be collected multiple times from the same chamber during an experiment. In a further embodiment, the bioreactor allows for multiple samples to be collected from multiple chambers during an experiment. In a further embodiment, these sample volumes allow for various analytical techniques and readouts including, but not limited to, untargeted mass-spectrometry (MS), targeted MS, or NMR-based metabolomic analysis; proteomic analysis (SDS-PAGE, Western blot, mass-spectrometry-based or other); transcriptional profiling (e.g., mRNA expression, miRNA expression, RNA-sequencing, ATAC-sequencing, reporter bioassay etc.); phenotypic bioassay; labeling of cells (e.g., fluorescent probes, antibody-based staining, viability markers), bacterial viability enumeration (e.g., colony forming unit counting), measuring change in pH, measuring change in redox potential, and more, either taken singularly or multiple times throughout the experiment.

In a further embodiment, the bioreactor allows for varied sample volumes due to replenishment of the cell culture environment. In a further embodiment, the smallest sample taken is one that is feasible to be measured for the desired analytical technique. In one embodiment, the bioreactor enables samples ranging in volume from microliters to 100 ml. In a further embodiment, the bioreactor allows for sample volumes up to about 25 ml to be collected. In a further embodiment, the bioreactor allows for sample volumes up to and including 100 ml at any given time, provided the cell culture has been replenished with media.

In one embodiment, the bioreactor is maintained at a temperature optimal for specific conditions. In one embodiment, the bioreactor is maintained at a temperature of 15-40 degrees Celsius, in particular 25-37 degrees Celsius. In another embodiment, the bioreactor is maintained at a temperature of 15-25 degrees Celsius. In another embodiment, the bioreactor is maintained at a temperature of 20-25 degrees Celsius. In another embodiment, the bioreactor is maintained at a temperature of 30-37 degrees Celsius. In one embodiment, the is maintained at room temperature. In another embodiment, the bioreactor is maintained at a temperature of 30 degrees Celsius. In another embodiment, the bioreactor is maintained at a temperature of 37 degrees Celsius.

In one embodiment, the bioreactor is maintained at ambient environment gas conditions. In another embodiment, the bioreactor is maintained with environment gas conditions between 0.1% O₂ v/v to about 21% O₂ v/v. In an embodiment, the bioreactor is maintained at ambient environment gas conditions (˜21% O₂ v/v) representing an aerobic environment or with anaerobic gas mix purging the bacterial chamber measuring steady oxygen levels as low as 0.1% O₂ v/v, or at a level in between.

In a further embodiment, cells can be added to either compartment within a chamber to facilitate different co-culture experiments.

The present invention comprises a bioreactor comprising cells and at least two separate chambers, wherein said chambers comprise cells and their own suitable media and environmental conditions favorable to cell growth, and wherein said chambers have their contents continuously mixing to expose a separate chamber comprising separate cells to the secreted products of the other chamber(s).

In one embodiment, the pumps are automated, electro-fluidic pumps, to transfer media to the different chambers. In one embodiment, the bioreactor system comprises a fraction collector. In one embodiment, the bioreactor system comprises an automated sample collection.

In one embodiment, the pumps are any suitable pump to transfer fluid. In another embodiment, the contents of the chambers or the samples taken from the chambers are via any suitable pump, including solenoid diaphragm pumps.

In one embodiment, the bioreactor comprises a multi-chamber cell culture system capable of emulating the gastro-intestinal tract. In an embodiment, the cell environments respond to each other based on secreted signals by a separate cell culture. In a further embodiment, cells respond by bioluminescence induction.

The present invention comprises a method of emulating the gastrointestinal tract, comprising culturing cells in a suitable medium in separate chambers of a bioreactor, wherein the bioreactor comprises a plurality of chambers, and wherein the chambers have one or more orifices through which a hollow conduit connects the chambers and allows them to be in fluid communication, wherein the contents from the chambers are mixed and circulated with the other chamber(s). In a further embodiment, the method comprises culturing mammalian epithelial cells in one chamber of a two chamber bioreactor and culturing microbial cells in a second chamber of the bioreactor, wherein both cell culture contents comprise secreted products from the cell culture are mixed and circulated between the two chambers. In a further embodiment, the chambers comprise a membrane, wherein the membrane divides the chamber into two sections (a section inside the membrane, and a section outside the membrane). In a further embodiment, the chambers comprise continuous mixing to expose the cell culture of each chamber to the secreted product of the other chambers enabling communication across the cells grown in separate environments, wherein the communication comprises the cells responding to the secreted signals upon co-culture.

The present invention comprises a method of emulating the gastrointestinal tract, comprising culturing cells in a suitable medium in a bioreactor system, wherein the bioreactor system comprises a plurality of chambers, wherein a first chamber comprises an inner membrane defining an inner volume and an outer wall surrounding the inner membrane and defining an outer volume surrounding the inner membrane, and a second chamber comprising an optional inner membrane defining an inner volume, and an outer wall surrounding the optional inner membrane, wherein if the inner membrane is present, then the outer wall defines an outer volume surrounding the inner membrane, and if the inner membrane is not present, then the outer wall defines an outer volume comprising all the contents in the chamber, wherein each chamber houses cells cultured in a suitable medium and environmental conditions, and wherein the chambers have one or more orifices through which a hollow conduit connects the chambers and allows them to be in fluid communication, wherein the contents from the chambers are mixed and circulated with the other chamber(s).

Interspecies interactions can be measured or tested in a variety of ways, including, but not limited to, untargeted mass-spectrometry (MS)-based, targeted MS-based, or NMR-based metabolomic analysis; proteomic analysis (SDS-PAGE, Western blot, mass-spectrometry-based or other); transcriptional profiling (e.g., mRNA expression, miRNA expression, RNA-sequencing, ATAC-sequencing, reporter bioassay etc.); phenotypic bioassay; labeling of cells (e.g., fluorescent probes, antibody-based staining, viability markers), bacterial viability enumeration (e.g., colony forming unit counting), measuring change in pH, measuring change in redox potential, and more. Most experimental tests require appropriate axenic controls (e.g., human cells only, bacterial cells only, human cells+bacterial cells).

The bioreactor may comprise any one of multiple sensors capable of detecting conditions in each chamber, but not limited to, oxygen concentration, pH and/or temperature.

In an embodiment, the bioreactor uses media optimal for cell growth. In one embodiment, bacterial media is selected from autoinducer bioassay (AB) medium (Boston Bioproducts, custom made to order and referenced in Greenberg et al., Arch. Microbiol. 120, 87-91 (1979)), brain heart infusion (BHI) medium (Anaerobe Systems), Yeast Casitone Fatty Acids Broth with Carbohydrates (YCFAC) (Anaerobe Systems). In an embodiment, human cell media is anything known in the art, RPMI, McCoy 5a, or Dulbecco's Modified Eagle Medium (DMEM) (Gibco) with 10% fetal bovine serum (FBS), and most preferably Dulbecco's Modified Eagle Medium (DMEM) (Gibco) with 10% fetal bovine serum (FBS) (Gibco).

The present invention includes a method for culturing two distinct cell populations simultaneously in a dual chamber bioreactor wherein each chamber houses a distinct cell population with its own favorable media and environment conditions, and wherein the supernatants in both chambers are continuously mixed to expose a separate chamber containing the a separate cell population to the secreted products of the other chamber.

The present invention includes a method for culturing two distinct cell populations simultaneously in a dual chamber bioreactor wherein each chamber is exposed to the secreted signals from the cells in the opposing chamber, and wherein the cells respond to the secreted signals.

### BRIEF DESCRIPTION OF THE DRAWINGS

FIG. 1A is a diagram representing a top-down view of the setup and design of an embodiment of the invention representing a multi chamber system comprising two chambers that are used to house two separate cell types.

FIG. 1B is a schematic diagram representing a dual chamber system containing two chambers, each comprising a membrane, with an exchange of contents from the two chambers.

FIG. 1C is a schematic diagram representing anaerobic or aerobic bacterial species in one chamber of the bioreactor and a human epithelial cell layer in the opposite chamber.

FIG. 1D is a schematic diagram representing a dual chamber system containing two chambers, wherein one chamber comprises a membrane and the second chamber does not comprise a membrane.

FIG. 2A is a picture of a side view of the setup and design of one aspect of the invention and represents a multi chamber system comprising two chambers that are used to house two separate cell types.

FIG. 2B is a schematic diagram representing a top down view of the setup of FIG. 2A.

FIG. 3A is a picture representing a bioreactor system that is placed on stage next to pumps and connected via silicone tubing.

FIG. 3B is a picture representing the pump functionality for initial experimental setup.

FIG. 4A is a line graph showing FD20 concentration in chamber 1 and chamber 2, both outside the membrane over 50 hours.

FIG. 4B is a schematic diagram representing the setup of a two chamber system as described in Example 1.

FIG. 5A is a line graph showing bioluminescence in chamber 1 and chamber 2, both outside the membrane.

FIG. 5B is a schematic diagram that represents the setup of a two chamber system as described in Example 2.

FIG. 6A is a line graph showing relative fluorescence units (RFU) of untreated, collagen-treated and collagent+Caco-2 membranes over 50 hours as represented in Example 3.

FIG. 6B is a bar chart showing after approximately 2 days, both the untreated and collagen-treated membranes reached near-equilibrium of 20 kD FITC-dextran (FD20) diffusion.

FIG. 7 is a representative image of one of the stitched images in Example 3.

FIG. 8A is a schematic diagram of the design and setup of Example 4 with two chambers.

FIG. 8B is a line graph showing the bioluminescence data of time-dependent supernatants from bioreactor in V. harveyi MM32 bioassay.

FIG. 9A is a line graph showing oxygen concentration measurements for approximately 3 days in 3 locations within the bioreactor system: i) chamber 1, outside the membrane, ii) chamber 1, inside the membrane, and iii) chamber 2, inside the membrane, and additionally an oxygen probe placed in the source media bottle 1.

FIG. 9B is a schematic diagram representing the setup in Example 5 where tubing connected a house nitrogen line to one PBS bottle (source media bottle 1).

FIG. 10A is a line graph representing the density of Fusobacterium nucleatum and Faecalibacterium prausnitzii as used in Example 6.

FIG. 10B is a schematic diagram representing the setup described in Example 6.

FIG. 10C is a line graph showing the results of Example 6.

FIG. 10D is a schematic diagram representing of Example 6 where BHI media was pumped into chamber 1 outside the membrane and PBS was pumped into chamber 2 outside the membrane.

FIG. 11A is a line graph showing the results of Example 7.

FIG. 11B is a schematic diagram representing the setup described in Example 7.

FIG. 12A is a schematic diagram representing the setup described in Example 8.

FIG. 12B is a schematic diagram representing the setup described in Example 8.

FIG. 12C is a schematic diagram representing the setup described in Example 8.

FIG. 12D is a schematic diagram representing a setup where multiple bacteria can be indirectly co-cultured in the bioreactor system with no human cells and separate media inputs.

FIG. 12E is a schematic diagram representing a setup where multiple bacteria can be indirectly co-cultured in the bioreactor system with no human cells and have the same media inputs.

FIG. 12F is a schematic diagram representing a setup where more than two bacteria chambers can be co-cultured in the bioreactor system.

FIG. 13A is a schematic diagram of one potential configuration of the bioreactor.

FIG. 13B is a schematic diagram of one potential configuration of the bioreactor.

FIG. 14A is a scatter plot showing the results of Example 7.

FIG. 15A is a schematic diagram representing anaerobic or aerobic bacterial species in one chamber of the microplate-based format bioreactor and a human epithelial cell layer in the opposite chamber.

FIG. 15B is a schematic diagram of one potential configuration of the microplate-based format bioreactor.

FIG. 15C is a rendered image of an example of the containment house used for the microplate-based format bioreactor.

FIG. 16A is a line graph showing the results of Example 10.

FIG. 16B is a schematic diagram representing the setup described in Example 10.

FIG. 17A is a line graph showing the results of Example 11.

FIG. 17B is a schematic diagram representing the setup described in Example 11.

FIG. 18A is a line graph showing the results of Example 12.

FIG. 18B is a schematic diagram representing the setup described in Example 12.

FIG. 18C is a line graph showing the results of Example 12.

FIG. 18D is a schematic diagram representing the setup described in Example 12.

### DEFINITIONS

As used throughout the specification and in the appended claims, the singular forms “a,” “an,” and “the” include the plural reference unless the context clearly dictates otherwise. Reference to “or” indicates either or both possibilities unless the context clearly dictates one of the indicated possibilities. In some cases, “and/or” was employed to highlight either or both possibilities.

A chamber is an enclosed space and/or is contained. Chamber 1 and chamber 2 are interchangeable and are modular.

The word ‘dual chamber’ refers to 2 or more chambers. Therefore, dual chambers can refer to 2 chambers, 3 chambers, 4 chambers and up to n number of chambers. Dual chambers can refer to 2 chambers. Dual chambers can refer to 3 or 4 chambers. Likewise, a multi-chamber system refers to 2 or more chambers.

Chambers are made up of vessels which can comprise any standard lab equipment, such as but not limited to a 50 ml conical tube or a microplate.

For clarification, a bioreactor system can comprise two chambers representing a single bioreactor, or a bioreactor system can comprise 2 bioreactors each comprising one chamber. Both concepts are interchangeable. A bioreactor system can also be referred to as a bioreactor.

The word ‘membrane’ is to be interpreted broadly to include such terms as dialysis bag, dialysis tube and represent a lining as representative in the gut or a physical barrier to restrict large particles or molecules while allowing smaller ones to diffuse through.

An ‘orifice’ is an opening, hole, or port.

A “conduit’ is a passage (a pipe, tunnel, tubing) through which water, liquids, or gases can pass.

The term ‘aerobic’ indicates the survival of cells in an environment with the presence of oxygen.

The term ‘anaerobic’ indicates the survival of cells in an environment without oxygen.

The word continuously mixing is to be interpreted broadly to include mixing co-cultures for a varied amount of time once the experiment has started to the end of the experiment. Such experiments can run for an hour and in theory an infinite amount of time as long as the cells continue to live. Realistically, the co-cultures could be mixed for the duration of an experiment, about 1-14 days. In particular, the supernatant or contents outside the membrane are mixed.

The word ‘fluid’ and the term ‘fluid material’ are to be interpreted broadly to include not only liquid and gas phase materials but also slurries that comprise solid or semi-solid material suspended in a liquid phase.

The term ‘feed material’ is to be interpreted broadly to include a liquid phase or a gas phase material or a slurry that comprises solids or semi-solids suspended in a liquid phase, and combinations of one or more phases thereof, which is used to facilitate the growth of cell or tissue cultures.

The words ‘cell culture’ or ‘cell medium’ are to be interpreted broadly to include any medium that facilitates the growth of cell and tissues.

The word ‘Epithelial culture’ is to be interpreted broadly to include any medium that facilitates the growth of Epithelial cell and tissues.

The word ‘Bacterial culture’ is to be interpreted broadly to include any medium that facilitates the growth of Bacterial cell and tissues including but not limited to a pure culture as in one species, a defined community as in a mixed species in the lab, or an undefined community as in a stool sample from a subject.

The word ‘co-culture’ is to be interpreted broadly to include exposing the opposing chamber containing the media, supernatants, with or without cells. ‘Opposing cells’ does not have to refer to a different cell type. Opposing cells merely refers to cells of a different chamber.

An anaerobic environment is one in which the air has been purged with anaerobic gas and contains steady oxygen levels as low as 0.1% O₂ v/v.

An aerobic environment is one at ambient environmental gas conditions (˜21% O₂ v/v).

The words ‘two separate cell cultures’ or ‘two separate cell populations’ can represent microbial and mammalian cells but can also represent mammalian and mammalian, or microbial and microbial. In this context, it is not limited to different but merely separate cell cultures in separate chambers. These separate chambers are joined via tubes to expose one chamber to the products of the opposing chamber.

The words ‘secreted products’ may include, but are not limited to, proteins (such as enzymes, antibodies, cytokines, chemokines, growth factors, hormones), peptides (such as antimicrobial peptides, peptide hormones, non-ribosomal peptides,), small molecules such as metabolites, hormones, amino acids, hormones, steroids, pheromones, lipids, bile acids, xenometabolites, biogenic amines, short-chain fatty acids, sugars, vitamins, polyamines, polyketides, etc.), nucleic acids (such as siRNA, sRNA, ncRNA, eDNA, ssDNA, etc.).

Abbreviations used are those conventional in the art of the following.
AB autoinducer bioassay
h hour
BHI Brain heart infusion
° C. degree Celsius
DMEM Dulbecco's Modified Eagle Medium
EtOH Ethanol
FBS Fetal Bovine Serum
FD20 20 kD FITC-dextran
FITC Fluorescein isothiocyanate
LB Luria-Bertani
kD kilodalton
min minute
ml milliliter
mM millimolar
nM nanomolar
PBS Phosphate-buffered saline
rfu relative fluorescence units
rpm revolutions per minute
% v/v percent volume
μl microliter
YCFAC Yeast Casitone Fatty Acids Broth with Carbohydrates

### DETAILED DESCRIPTION OF THE INVENTION

The present invention includes a bioreactor for co-culturing cells, wherein the bioreactor comprises bacterial cells and human cells, wherein the cells are grown in separate chambers within a bioreactor system, and wherein each chamber comprises favorable media and/or environmental conditions, and wherein each chamber is separated into two sections by a membrane, wherein the contents from one section of the chamber comprising secreted products are continuously mixed to expose the cells of each chamber to the secreted products of the other chamber. Upon co-culture, cells can sense and respond to these secreted signals, continuously shaping the secreted dialogue.

In an embodiment, cell free collection is removed from a top port of the chamber and does not reenter the system.

In one embodiment, a chamber comprising bacterial cells comprise a mucin-coated membrane.

In one embodiment, chamber 1 comprises a bacterial culture, wherein bacterial cell media is supplied into the mucin-coated membrane through a septum on the lid and a syringe.

In one embodiment, chamber 2 comprises an epithelial culture, wherein human cell media is supplied directly to the basal side of cells through a top port which enters the chamber by a tube outside of a collagen coated membrane.

In a further embodiment, continuous mixing of secreted bacterial products from chamber 1 and from the apical side of cells from chamber 2 is performed through the top ports of each chamber. In an embodiment, secreted bacterial products from outside the membrane are removed from chamber 1 and entered into chamber 2 membrane. In another embodiment, cells located in the collagen-coated membrane of the epithelial culture are removed from chamber 2 and entered into the outside of the mucin-coated membrane in the bacterial cell chamber.

One aspect of the present invention is the setup of the chambers contained within the bioreactor system, wherein the bioreactor system is not limited to bacterial species in one chamber of the bioreactor system and a human epithelial cell layer in the neighboring chamber. In one embodiment, other human cell types in the unit are located basally to a epithelial cell layer, wherein other human cell types comprise immune cells, endothelial cells, fibroblasts, endocrine cells, neuronal cells, adipocytes, podocytes, muscle cells, or other cell types.

In one aspect of the invention, two centrifuge tubes represent a multi-chamber system comprising two chambers and are used to house two separate cell types. In one embodiment, the first centrifuge tube (the first chamber), located on the left, houses the first cell type (e.g., bacterial culture), and the second centrifuge tube, the second chamber, houses the second cell type (e.g., epithelial culture). In one embodiment, the centrifuge tubes, wherein the centrifuge tubes represent chambers, are interchangeable.

In another aspect of the invention, two microplates represent a multi-chamber system comprising two chambers and are used to house two separate, but not necessarily distinct, cell types. In another aspect of the invention, each microplate representing chamber can comprise multiple wells.

In another aspect of the invention, one well on a microplate represents a chamber and either i) two wells on two separate microplates, or ii) two wells on the same microplate represents a multi-chamber system. In another embodiment, the microplate is within a containment housing. In an embodiment, the contents of one chamber in a microplate in a containment housing can be exchanged with the contents of a chamber in a microplate in an adjacent containment housing. In another embodiment, the contents of one chamber in a microplate in a containment housine can be exchanged with the contents of a chamber on the same microplate in the same containment housing.

In one aspect of the invention, inside each tube is a membrane with a threaded crown. In one embodiment, the membrane is uncoated or coated with an extracellular matrix component. In yet a further embodiment, the coating is collagen. In one embodiment, the epithelial cell membrane is coated with collagen to enhance cell adherence. Collagen is an extracellular matrix (ECM) component, allowing cells to adhere to it—it is likely other ECM substances can promote adherence, or other surface treatments for enhanced cell adherence (e.g., poly-L-lysine). In one embodiment, surface coatings, such as mucin, can also be used in the membrane that contains bacteria to provide additional physiological cues.

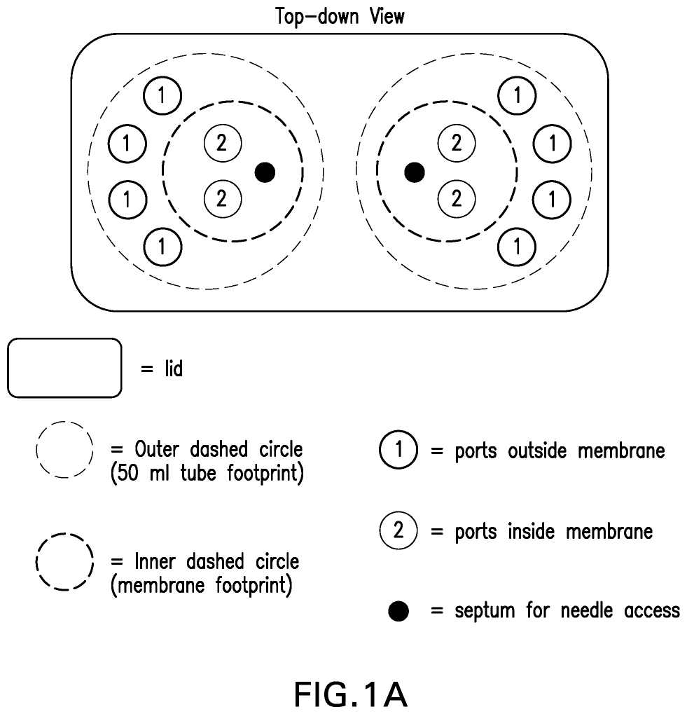

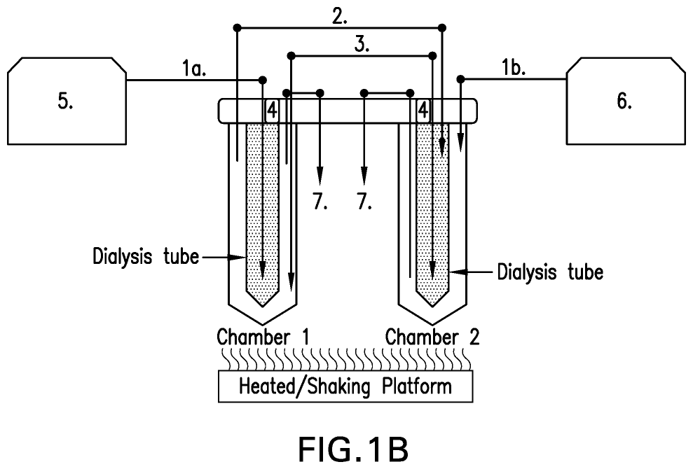

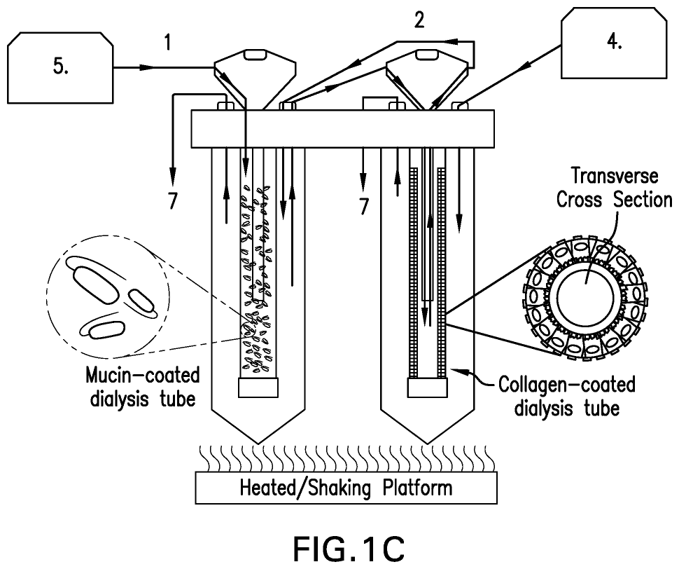

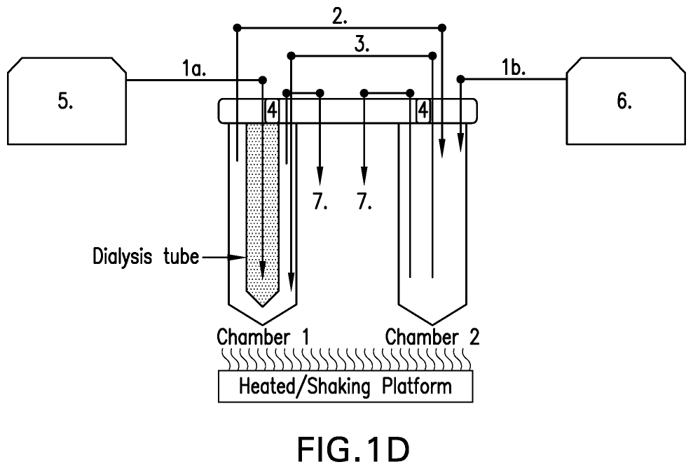

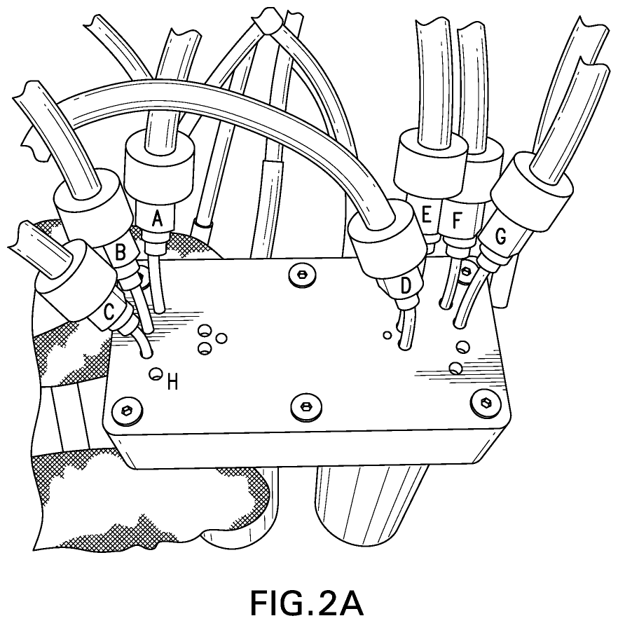

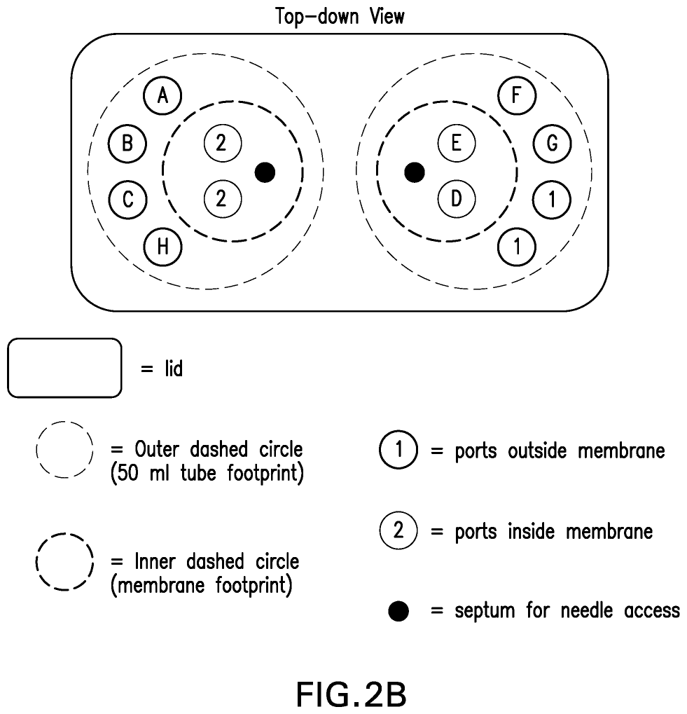

In one aspect of the invention, both the chambers and threaded membrane are screwed into a custom autoclavable 3D printed sintered nylon lid (lid′). In one embodiment, the lid has seven access ports (orifices), as shown in FIG. 1A, for each centrifuge tube. In a further embodiment, four access ports on the lid go the outside of the membranes for plumbing. As shown in FIG. 1, the four access ports on the lid outside of the membrane are represented by four circles located in the outer circle as shown by the circle with ‘1’ inside. In another embodiment, two access ports on the lid go to the inside of the membrane for plumbing. FIG. 1 represents the two access ports for the inside of the membrane as shown by the circle with ‘2’ inside. In another embodiment, one port on the access lid is sealed with a silicone septum that can be accessed using a needled syringe. In a further embodiment, this port accessed using a needled syringe is used for sample removal and/or bacterial inoculation. FIG. 2A represents ports labelled A-G, and the ports used for access to the inside of the membrane are interchangeable with one another, whereas the ports used for access to the outside of the membrane are interchangeable with one another.

FIG. 1A is a schematic diagram that represents a top-down view of the setup and design of an embodiment of the invention representing a multi chamber system comprising 2 chambers that are used to house two separate cell types. This top-down view of the ports (orifices) located in a two-chamber system is not drawn to scale and is used for illustrative purpose to represent where each port could be located relative to the location of the membrane mounted inside the tube. Variations in the port locations, not limited to location on the lid or distance from the center or from other port locations, is expected.

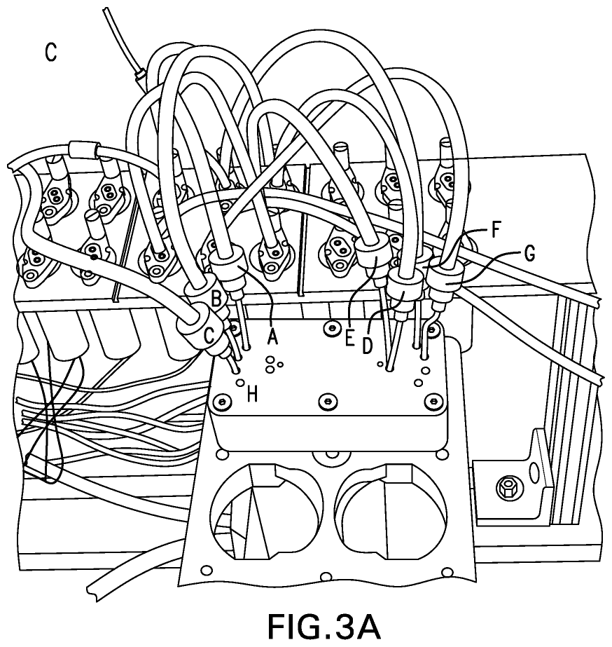

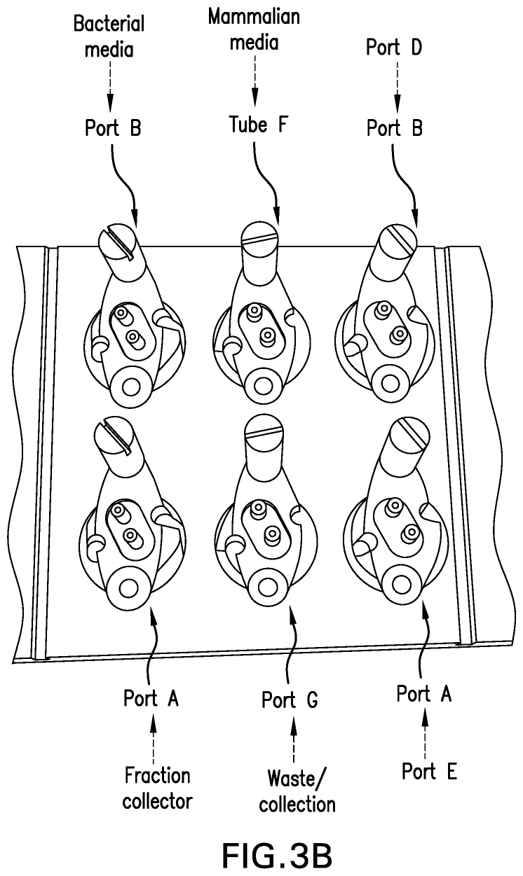

FIG. 1B is a schematic diagram representing a dual chamber system containing two chambers, each comprising a membrane, with an exchange of the contents inside or outside the membrane in each compartment. In this diagram, the contents outside the membrane in chamber 1 is exchanged to the inside of the membrane in chamber 2, and the contents inside the membrane in chamber 2 are exchanged to the contents outside of the membrane in chamber 1. Arrowed lines on schematic are representative of the fluidic tubing (and flow direction) on the apparatus and are not drawn to scale. The following numbers on FIG. 1B are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Cell media source into chamber 1 (Source Media Bottle).
6. Cell media source into chamber 2 (Source Media Bottle).
7. Outputs for sampling.
   FIG. 1C is a schematic diagram representing anaerobic or aerobic bacterial species (either individual strains or communities of multiple strains) in one chamber of the bioreactor system and a human epithelial cell layer in the neighboring chamber. In this diagram, epithelial cells are used for interkingdom (e.g. eukaryotic-prokaryotic) communication and also for oxygen barrier/sequestration. This figure is used for illustrative purposes to represent the cells in each chamber, and as an example, a bacterial chamber and a epithelial chamber comprising human cells. Chamber 1 is a bioreactor comprising a bacterial culture comprising a membrane, wherein the membrane is a dialysis tube. Chamber 2 is a bioreactor comprising an epithelial culture comprising a membrane, wherein the membrane is a collagen-coated dialysis tube. Human cell media is supplied directly to the basal side of the cells. The following numbers on FIG. 1C are identified as the following:
1. Arrowed lines on schematic are representative of the fluidic tubing (and flow direction) on the apparatus and are not drawn to scaleMedia input into chamber 1—Anaerobic media supplied to bacteria
2. Continuous mixing of secreted bacterial products and apical side of cells.
4. Cell media source into chamber 2—Human Cell Media
5. Cell media source into chamber 1—Bacterial cell media
7. Outputs for sampling.
   FIG. 1D is a schematic diagram representing a dual chamber system containing two chambers, wherein one chamber comprises a membrane and the second chamber does not comprise a membrane. In such an example, there could be an exchange of contents from the inside or outside of the membrane in chamber 1 to chamber 2, and back from chamber 2 to the inside or outside of the membrane of chamber 1. In this figure, the contents outside the membrane in chamber 1 is exchanged to chamber 2, and the contents from chamber 2 are exchanged to the contents outside of the membrane in chamber 1. Arrowed lines on schematic are representative of the fluidic tubing (and flow direction) on the apparatus and are not drawn to scale. The following numbers on FIG. 1C are identified as the following:
1a. Media input into chamber 1.
1b. Media input into chamber 2
2. Exchange of contents from chamber 1 into chamber 2 through a hollow conduit.
3. Exchange of contents from chamber 2 into chamber 1 through a hollow conduit.
4. Septum port/valve (orifice).
5. Cell media source into chamber 1 (Source Media Bottle).
6. Cell media source into chamber 2 (Source Media Bottle).
7. Outputs for sampling.
   FIG. 2A represents a side view of the setup and design of one aspect of the invention and represents a multi chamber system comprising two chambers that are used to house two separate cell types. Two 50 ml tubes (chambers) are attached underneath a lid which contains ports (orifices) and tubing (hollow conduit) labelled A to G. The ports are represented in FIG. 1A and again in FIG. 2B to match the tubing descriptions as shown in FIG. 2A.
   FIG. 2B is a schematic diagram representing a top-down view of the setup of FIG. 2A. Ports (orifices) ‘A’, ‘B’, ‘C’, and ‘H’ represented on the lid of the left chamber are all ports located outside the membrane. Ports ‘F’ and ‘G’ represented on the lid of the right chamber are ports, along with 2 additional ports labelled as ‘1’, are all ports located outside on the membrane. FIG. 2B represents ports labelled ‘1’ and ‘2’ including black dots for septum ports.
   FIG. 3A is a picture representing a bioreactor system that is placed on stage next to pumps and connected via silicone tubing. Port C is connected to the anaerobic gas mix. Port H, which is a port with no tubing connected, is used to insert a needle with 0.2 μm filter to relieve pressure from the incoming gas.
   FIG. 3B is a picture representing the pump (Solenoid diaphragm pump (Lee Co, LPLA2451650L) functionality for initial experimental setup. There are 6 pumps. The first pump represents the bacterial media going into Port B. The second pump represents the mammalian media going into Tube F. The third pump represents Port D to Port B. The fourth pump represents Port A to fraction collector. The fifth pump represents Port G to Waste/Collection. The sixth pump represents Port A to Port E. FIG. 3B represents the solenoid diaphragm pump (Lee Co, LPLA2451650L) positioning for one variety of an initial experimental setup. FIG. 3A represents a bioreactor system that is placed on stage next to pumps and connected via silicone tubing, wherein the pumps are solenoid diaphragm pumps (Lee Co, LPLA2451650L). In one embodiment, one port located outside the membrane, is connected to the anaerobic gas mix. In one embodiment, one port located outside the membrane, is a port wherein no tubing is connected, and used to insert a needle with 0.2 μm filter to relieve pressure from the incoming gas. In one embodiment of the invention, there are 6 pumps wherein pump 1 represents the bacterial media going into port B, pump 2 represents the mammalian media going into tube F, pump 3 represents port D to port B, pump 4 represents port A to fraction collector, pump 5 represents port G to waste or collection, and pump 6 represents port A to port E. In one embodiment, diaphragm pumps control the fluid inputs, mixing, and outputs. In a further embodiment, the fluid is media. In one embodiment, outputs are cell-free and collected automatically using a fraction collector with 3-way diverter. In one embodiment, the pumps are interchangeable.

In one embodiment of the invention, the bioreactor system sits on a heating plate. In one aspect of the invention, the bioreactor system sits on a shaking plate. In a further embodiment, the bioreactor system sits on a heating and shaking plate. In a further embodiment, the heating and shaking plate is an Inheco unit. In a further embodiment, all components comprising pumps, heating and shaking plate, fraction collection are integrated and controlled by one software package. In a further embodiment, the software package is custom Telios software package.

In another embodiment, each chamber has a volume range of 350 μl to 50 ml, preferably 3 ml to 50 ml. In one aspect of the invention, each compartment has a maximum volume of about 50 ml. In one embodiment, each chamber is approximately 3 ml. In another embodiment, each chamber is 350 μl.

In one embodiment, the bioreactor system comprises at least one sampling orifice in fluid communication with the inner volume, and wherein the at least one sampling orifice allows removal of fluid from the inner volume.

In one embodiment, the bioreactor system comprises at least one sampling orifice in fluid communication with the outer volume, and wherein the at least one sampling orifice allows removal of fluid from the outer volume.

In one embodiment, the bioreactor system comprises at least one sampling orifice in fluid communication with the inner volume, and wherein the at least one sampling orifice allows insertion of fluid to the inner volume.

In one embodiment, the bioreactor system comprises at least one sampling orifice in fluid communication with the outer volume, and wherein the at least one sampling orifice allows insertion of fluid to the outer volume.

In one embodiment, the bioreactor system comprises at least one sampling orifice to the inner membrane. In one embodiment, the bioreactor system comprises at least one sampling orifice to the outer membrane. In one embodiment, the bioreactor system comprises two sampling orifices to the inner membrane. In one embodiment, the bioreactor system comprises four sampling orifices to the outer membrane. In one embodiment, an orifice is a port.

In an embodiment of the invention, a bioreactor comprises two compartments. In one embodiment, two compartments are connected to represent a bioreactor with a maximum volume of 100 ml. In a further embodiment, a bioreactor comprising two compartments comprises a working volume of about 80 ml. This 80 ml working volume is significantly larger than previously bioreactors (von Martels et al., Anaerobe 44 (2017) 3-12). In one embodiment, the bioreactor comprises a volume that allows sampling over a period of time, wherein this sampling allows chemical or bioassay analysis throughout the duration of the bioreactor process.

In one aspect of the invention, the modular design of the bioreactor system comprises multiple bioreactors.

In one aspect of the invention, dialysis bags or dialysis tubes of different pore size can be leveraged for size-based selection of secreted factors to be shared within the cell culture in the other chamber.

In one aspect of the invention, the liquid in each chamber is transferred via pumps in hollow conduits. In one aspect of the invention, each chamber comprises a lid, wherein the lid comprises orifices.

### Materials

Standard 50 ml centrifuge tubes (Nunc, Thermo Scientific, 339653)
Autoclavable 3D printed sintered nylon lid (Clickbio, Inc custom part)
Autoclavable custom aluminum containment housing (Clickbio, Inc custom part)

### Pumps and Pump Modules

Solenoid pump (Lee Co, LPLA2451650L)
Pump module (Biomated Solutions, MWP004)
Ismatec IPC 12 channel peristaltic pump (78001-20)

### Chamber Stage, Heating/Agitation

Thermoshake (Inheco, 7100146)
Single TEC Control (Inheco, 8900031)
6 position heater block (Clickbio, custom solution, www.click-bio.com/custom-solutions-shop/6-position-heater-block)

### Tubing

¼″ ID× 7/16″ OD PVC Tubing (Thermo Scientific, 8000-0070)
0.062 MINSTAC Tubing (TUTB32169200, The Lee Co.)
0.031 Masterflex L/S Platinum-Cured Silicone Tubing (Cole-Parmer, EW-96410-13)
⅛″ ID×¼″ OD Platinum-Cured Silicone Tubing (Cole-Parmer, #EW-95802-05)
0.062 MINSTAC Tubing Union (TMUA3201950Z, The Lee Co.)
Silicone tubing, platinum cured, ID 0.64 mm (Cole Parmer, 95602-22)
Pharmed® BPT 0.25 mm ID tubing (Ismatec, 95723-12)
Stainless steel tubing, 1.58 mm OD, 1.23 mm ID, 50 mm length (Clickbio, Inc. custom part)

### Tubing Connections

¼″ to ⅛″ ID Reducer Barb (McMaster-Carr, 53055K516)
⅛″ to 1/16″ ID Reducer Barb (McMaster-Carr, 53055K514)
Polycarbonate Luer Fittings (Cole-Parmer, 45504-00)
062 MINSTAC-LFA Tubing Adapter (TMDA3207950Z, The Lee Co.)
TaegaSeal PTFE Tape (TaegaTech, Mil-T-27730A)
Flangeless Fitting (XP-283, IDEX Health & Science)
Flangeless Ferrule (UX-02007-54, Upchurch Scientific)
0.062 MINSTAC Union (TMUA3201950Z A, The Lee Co.)
0.062 MINSTAC Adapter (TMDA3201950Z, The Lee Co.)

### Anaerobic Gas Control and Monitoring

Omega In-Line Pressure Regulator (Omega, FMA-2620A)
Oxy-4 ST Trace (PreSens, S/N: SABU0002000008)
Needle-Type Housing Fiber-Optic Oxygen Microsensor (PreSens, NTH-PSt7-02-L2.5-TF-NS40-0.8-OIW)
Oxygen Dipping Probe (Presens, DP-PSt7-10-L2.5-ST10-YOP)
Pt100 Temperature Probe (PreSens, TEP-(FTC)-L5-St40-OD1.9 (for Fibox 4, Microx 4))

### Media, Additives, and Bottle Parts

Antifoam-204 (Sigma-Aldrich, A8311-50ML)
EC-Oxyrase (Oxyrase Inc., SAE0010-100ML)
¼-28 NAT PTFE Port Plugs (Cole-Parmer, 12020-47).
5/16″ Threaded PEEK Plug (Upchurch Scientific, 05-701-443)
Autoinducer bioassay (AB) medium (Boston Bioproducts, custom made to order)
Brain heart infusion (BHI) medium (Anaerobe Systems)
Yeast Casitone Fatty Acids Broth with Carbohydrates (YCFAC) (Anaerobe Systems)
Dulbecco's Modified Eagle Medium (DMEM) (Gibco)
Fetal bovine serum (FBS) (Gibco)
Phosphate buffered saline (Boston Bioproducts)

### Miscellaneous

Spline wrench (TTTA3200543C, The Lee Co.)
22Gx4″ Hypodermic Needles (Air-Tite Products Co. Inc, SKU: N224)
Norm-Ject Luer Slip Syringe (Air-Tite Products Co. Inc, SKU: A1)
Costar® Transwell® 6-well plate inserts (Corning, 3450)

### Automated Sample Collection

FC 204 Fraction Collector (Gilson, 171041)
508 Interface Module, 110-220 Volt (Gilson, 361832)

### System Monitoring and Control

Telios software package (version 20180201)

### System Containment

Aluminum T slotted extrusion framing and clear polycarbonate sheets compose the containment structure. D=2′, W=6′, H=3′ (not including filter unit). Front Access—One set of double doors 3.5′ opening to fluidics and vessels, single door access 2.5′ opening to fraction collection station.

### Example 1: Model Dye Injected in One Chamber can be Detected Throughout the Bioreactor System Over Time

Injection of model dye was tracked throughout the bioreactor system in a time-dependent manner.

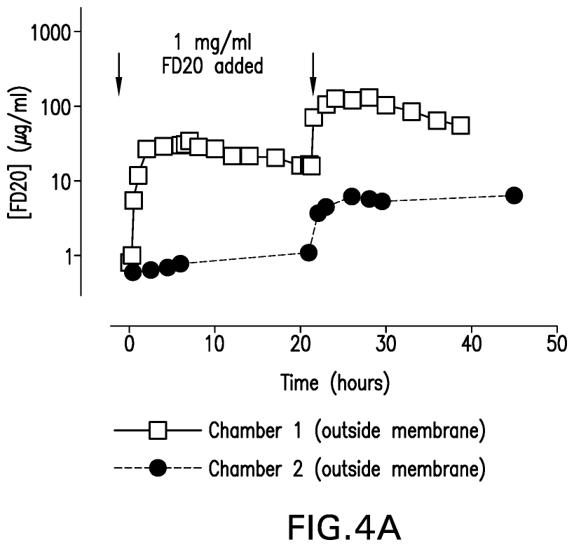

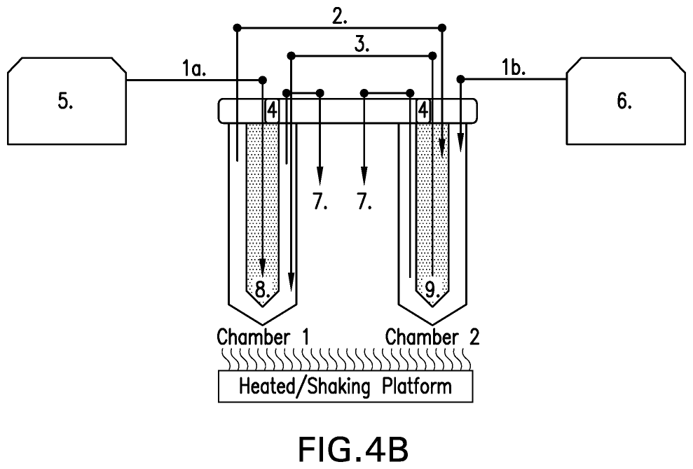

A membrane (1000 kD, Spectrum Labs, G235062) was attached to custom lid and 3.5 ml of 1 mg/ml 20 kD FITC-dextran (FD20) was filled inside the membrane in chamber 1 and 3.5 ml of water was filled inside of the membrane in chamber 2. Chambers were filled with 30 ml of water outside of the membrane. Fluidic pumps were turned on so that water was being added into and out of the chamber at 0.1 ml/min. Two fluidic pumps were used to circulate the water from outside the membrane in chamber 1 to inside the membrane in chamber 2 (and the reverse direction from the inside the membrane in chamber 2 to outside the membrane in chamber 1) at 1 ml/min. Samples coming from chamber output pumps were collected at predetermined time intervals. A second bolus of 350 μl 10 mg/ml FD20 was injected at 21 h. The system was ran for 48 h. Output samples, along with known standards of FD20, were loaded on flat, black, opaque bottom 96-well plates (Costar) and fluorescence was measured using PHERAstar® plate reader (480/520). FIG. 4A is a line graph showing FD20 concentration in chamber 1 and chamber 2, both outside the membrane over 50 hours. Chamber 1 is represented by the top data set (□) and chamber 2 is represented by the bottom data set (•). FIG. 4A corresponds with Example 1. As shown in FIG. 4A, fluorescence measurement was used to calculate concentration of FD20 in different locations of the bioreactor system over time. The location directly proximal to initial input of FD20 (i.e. chamber 1, outside membrane) shows higher fluorescence in comparison to the location furthest from FD20 input (i.e. chamber 2, outside membrane). Increases in FD20 concentration in chamber 1 were seen over time due to diffusion through membrane until a peak is reached, along with a slow decay due to perfusion. Gradual increases in FD20 concentration in chamber 2 were seen with a spike in concentration at the time of the second FD20 addition (21 h). FIG. 4B represents the setup of the bioreactor used in Example 1 for running the experiment. FIG. 4B is a schematic diagram representing a two-chamber system as described in Example 1. The dual chamber system comprises H₂O supplied to the outside of the membrane in chamber 1 and the outside of the membrane in chamber 2. Arrowed lines on schematic are representative of the fluidic tubing (and flow direction) on the apparatus and are not drawn to scale In this example, number 9 is blank. The following numbers on FIG. 4B are identified as the following:
1a. Media input into chamber 1.
1b. Media input into chamber 2
2. Exchange of contents from chamber 1 into chamber 2 through a hollow conduit
3. Exchange of contents from chamber 2 into chamber 1 through a hollow conduit
4. Septum port/valve (orifice)
5. Cell media into chamber 1 (H₂O)
6. Cell media into chamber 2 (H₂O)
7. Outputs for sampling.
8. FD20 dye added
9. Distinct cell culture or blank.

### Example 2: Kinetic Measurement of a Secreted Bacterial Product (E. coli and Blank), which is Representative of Secretion from One Chamber to the Other

A model organism was grown in the bioreactor and metabolite (dihydroxypentanedione) production was detected in multiple chambers via bioassay.

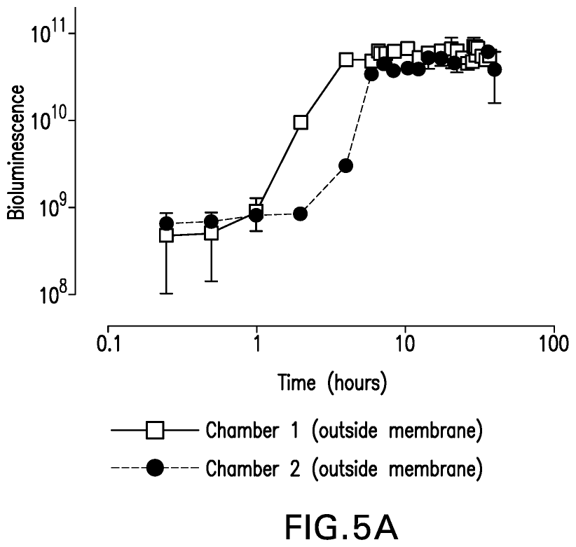

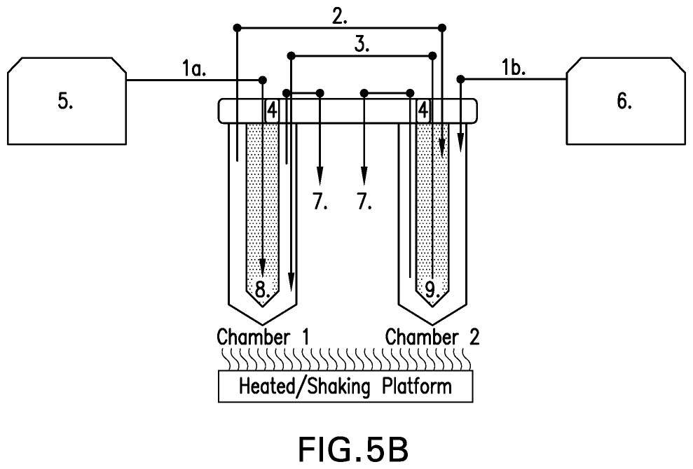

The bioreactor system was sterilized by autoclaving. Escherichia coli Top10 (LifeTechnologies) was streaked onto Luria-Bertani (LB) agar. A single colony was selected and used to inoculate 5 ml of LB broth and incubated at 37° C. overnight. FIG. 5B is a schematic diagram representing a two-chamber system as described in this example. The bioreactor system comprises Luria-Bertani (LB) media supplied to chamber 1 and LB Media supplied to chamber 2. Arrowed lines on schematic are representative of the fluidic tubing (and flow direction) on the apparatus and are not drawn to scale. The following numbers on FIG. 5B are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Cell media into chamber 1 (LB Media).
6. Cell media into chamber 2 (LB Media).
7. Outputs for sampling.
8. Distinct cell culture or blank for control (E. Coli).
9. Distinct cell culture or blank for control (blank).

As shown in FIG. 5B, the bioreactor system was assembled with two chambers, each with an EtOH-sterilized membrane attached inside. Chamber 1 membrane was filled with 4 ml of LB immediately before attaching to bioreactor system. LB media was pumped into each chamber outside the membrane until the entire membrane was covered (˜33 ml/chamber). Fluidics pumps were initiated at flow rates of 0.1 ml/min into and out of each chamber and 0.25 ml/min back-and-forth between the outside of the membrane in chamber 1 and inside of membrane in chamber 2. Shaking was set at 160 rpm and temperature was set to 37° C. The overnight culture of E. coli was diluted 1:10 in fresh LB media, and 1 ml of the diluent was injected with needled syringe through the rubber septum into the membrane of chamber 1. Samples were collected on 96-well deep well plates via fraction collector at predetermined timepoints for ˜40 h. Plates with samples were stored at −80° C. until use in downstream bioassay.

A small swab of frozen Vibrio harveyi MM32 (ATCC BAA-1121) in 30% glycerol was used to inoculate 5 ml of marine medium (Boston Bioproducts) and was incubated overnight at 30° C. The overnight culture was diluted 1:1000 in autoinducer bioassay (AB) medium (Boston Bioproducts) with 0.1 mM boric acid (Sigma) to make the working solution. All samples collected from both chambers of the bioreactor were diluted in the working solution at a final concentration of 10% v/v. A standard curve of known concentrations of dihydroxypentanedione (DPD; Cayman) was used. Aliquots of 100 μl of these working solutions with analyte were distributed on a 96-well plate. The plate was placed in a Biotek Synergy Neo2 multi-mode reader set at 30° C. Every half hour the plate was shaken for 10 seconds and then absorbance (600 nm) and luminescence were measured. FIG. 5A is a line graph showing Bioluminescence in chamber 1 (□) and chamber 2 (•), both outside the membrane. In FIG. 5A, the data are displayed as the bioluminescence signal normalized to reporter bacterial growth (A600) at the 8 h timepoint.

FIG. 5A displays DPD levels were below limit of detection by the biosensor strain in both chambers in the first 1 h of collection. At 2 h, DPD could be detected outside the membrane in Chamber 1 but still not detectable in Chamber 2. At 4 h, DPD was reaching signal saturation outside the membrane in Chamber 1 and was detectable outside the membrane in Chamber 2. By 6 h, DPD level outside the membrane in Chamber 2 had approached signal saturation. Signal saturation is typically reached at ˜100 nM and the lower limit of DPD detection with this biosensor strain in this assay format is ˜0.1-1 nM.

### Example 3: Epithelial Cells Grown on Membrane Scaffold

A series of bioreactors were used in this example.

Caco-2 cells were grown in a monolayer on membrane and displayed epithelial barrier function.

Membranes (1000 kD, Spectrum Labs, G235062) were completely immersed in water for ˜1 min to wash. The membranes were removed, the excess water was poured off, and the membranes were completely immersed in molecular biology grade 200 proof ethanol (Fisher Scientific) for 1 min. The membranes were removed, EtOH was removed by aspiration and the membranes were allowed to air dry for 30 min in a biosafety cabinet.

For collagen-coated membranes, the inside of the membranes were treated with 150 μg/ml collagen (Gibco, A1048301) in sterile PBS with 20 mM acetic acid (Sigma). The membranes were placed in a 37° C. incubator for 3 h. The membranes were removed, collagen solutions were aspirated, and membranes were washed with sterile PBS. The collagen-coated membranes in the chamber comprising Caco-2 cells were stored in 50 ml centrifuge tubes at 4° C. until use. The inside of the membranes were seeded with 5 ml Caco-2 cells (1e6 cells/ml) in DMEM with 10% FBS and penicillin/streptomycin. The sections outside of the membranes \were filled with ˜33 ml of the same media. The inside of the membranes were sealed using a threaded cap to prevent cells from leaking out. Tubes were placed on a tube roller in a 37° C., 5% CO₂ incubator, and the tube roller was initially set at 30 rpm. Rotational speed was steadily reduced at ˜1 rpm/sec until 0.5 rpm was reached. The tubes were left rolling for 24 h. After 24 h, the tubes were placed upright in a 37° C. 5% CO₂ incubator, and the caps were loosened to allow air flow. Three days after the cells were seeded, the media was aspirated from outside of the membrane and replaced with fresh media. Seven days after the cells were seeded, media was removed by aspiration from all tubes (inside and outside of membrane). The solution inside of the membranes was replaced with media+1 ug/ml FITC-dextran (20 kD; FD20). The solution outside of the membranes was replaced with fresh media. Aliquots (175 μl) were removed from the outside of the membranes at various timepoints up to 48 h. At the final timepoint, aliquots were also removed from inside the membranes to give a comparison across compartments at this timepoint. Samples were loaded onto black, opaque, flat bottom 96-well plate in duplicate and fluorescence intensity (485 ex, 516 em, gain 25) was measured on a Biotek Synergy Neo2 multi-mode reader.

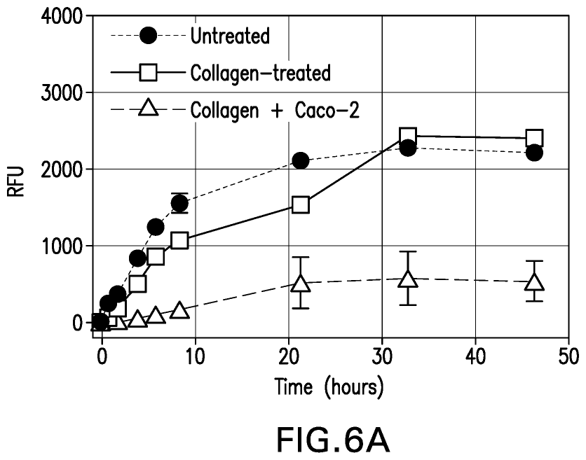

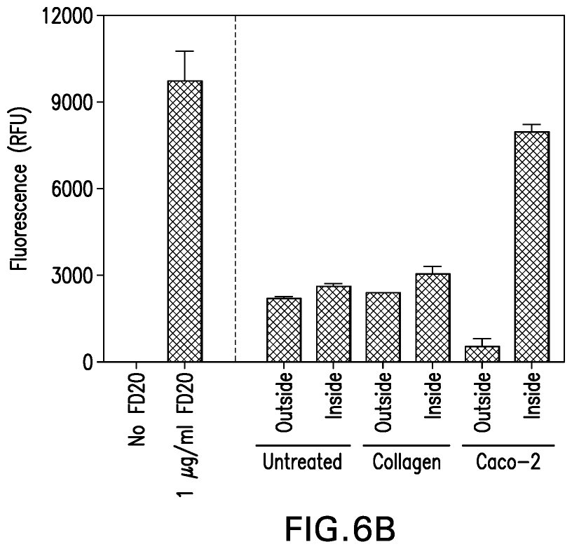

FIG. 6A is a line graph showing the relative fluorescence units (RFU) of untreated, collagen-treated and collagen+Caco-2 membranes over 50 hours as represented in Example 3. An exponential, one-phase association pattern is observed with the untreated and collagen-treated membranes. Untreated membranes approach equilibrium most rapidly, followed by collagen-coated membranes. The membranes that were coated with collagen and had Caco-2 cells growing on them showed a much slower approach toward equilibrium and showed significantly higher relative barrier function. The collagen-treated membranes were a control. FIG. 6A shows an exponential, one-phase association pattern is seen with the untreated and collagen-treated membranes. Untreated scaffolds approach equilibrium quicker than collagen-coated membanes and collagen-coated membranes with Caco-2 cells growing on them. The collagen-coated membranes containing no cells were next to reach equilibrium and in this experiment are used as a control. The membranes that were coated with collagen and had Caco-2 cells growing on them showed a much slower approach toward equilibrium and showed significantly higher relative barrier function. FIG. 6B is a bar chart showing that after ˜2 days (46.25 h), both the untreated and collagen-treated membranes reached near-equilibrium of 20 kD FITC-dextran (FD20) diffusion. In contrast, the Caco-2-seeded membranes had distinctly separated environments with very different levels of FD20, indicating that the cell layer forms a formidable barrier to FD20 diffusion, even over the course of ˜2 days. FIG. 6B, shows after approximately 2 days (46.25 h), both the untreated and collagen-treated membranes reached near-equilibrium of FD20 diffusion. In contrast, the Caco-2-seeded membrane had distinctly separated environments with very different levels of FD20, indicating that the cell layer forms a formidable barrier to FD20 diffusion, even over the course of ˜2 days.

In a separate experiment, membranes (1000 kD, Spectrum Labs, G235062) were completely immersed in water for ˜1 min to wash. The membranes were removed, the excess water was poured off, and the membranes were completely immersed in molecular biology grade 200 proof ethanol (Fisher Scientific) for 1 min. The membranes were removed, EtOH was removed by aspiration and the membranes were allowed to air dry for 30 min in a biosafety cabinet. For collagen-coated membranes, the inside of the membranes were treated with 150 ug/ml collagen (Gibco, A1048301) in sterile PBS with 20 mM acetic acid (Sigma). The membranes were placed in a 37° C. incubator for 3 h. The membranes were removed, collagen solutions were aspirated, and the membranes were washed with sterile PBS. The collagen-coated membranes were stored in 50 ml centrifuge tubes at 4° C. until use. The inside of the membranes were seeded with 5 ml Caco-2 cells (1e6 cells/ml) in DMEM with 10% FBS and penicillin/streptomycin. The sections outside of the membranes were filled with ˜33 ml of the same media. The inside of the membranes were sealed using a threaded cap to prevent cells from leaking out. Tubes were placed on a tube roller in a 37° C., 5% CO₂ incubator and tube roller was initially set at 30 rpm. Rotational speed was steadily reduced at ˜1 rpm/sec until 0.5 rpm was reached. The tubes were left rolling for 24 h. After 24 h, the tubes were placed upright in a 37° C. 5% CO₂ incubator, and the caps were loosened to allow air flow. Three days after the cells were seeded, the media was aspirated from outside of the membrane and replaced with fresh media. Seven days after seeding Caco-2 cells, the membranes were removed and tops and bottoms of the membranes were cut off using scissors, leaving a cylindrical membrane structure intact. In 15 ml tubes with approximately 10 ml volume per tube, membranes with cells were fixed with 4% paraformaldehyde in PBS (Boston Bioproducts). The membranes with cells were washed with PBS, and cells were permeabilized with 0.5% TritonX-100 (Sigma) in PBS for 10 min. The membranes with cells were washed with PBS, and the cells were blocked with 5% BSA in PBS (Sigma) for 1 h. The membranes with cells were washed with PBS, and the cells were labeled with 1 ug/ml Occludin monoclonal antibody (OC-3F10) AlexaFluor 488 (Thermo 331588) for 3 h. The membranes with cells were washed with PBS, and the cells were labeled with 2.5 U/ml Phalloidin CF633 (Biotium #00046) for 20 min. The membranes with cells were washed with PBS, and the cells were labeled with 300 nM DAPI (Invitrogen D1306) for 5 min. The membranes with cells were washed with PBS. The membranes with cells were cut down the middle into two pieces and were unfolded onto glass slides. The membranes with cells were mounted using ProLong Gold Antifade Mountant (ThermoFisher P36930) and stored in the dark overnight. Images were taken using the automated function on the EVOS fluorescence microscope

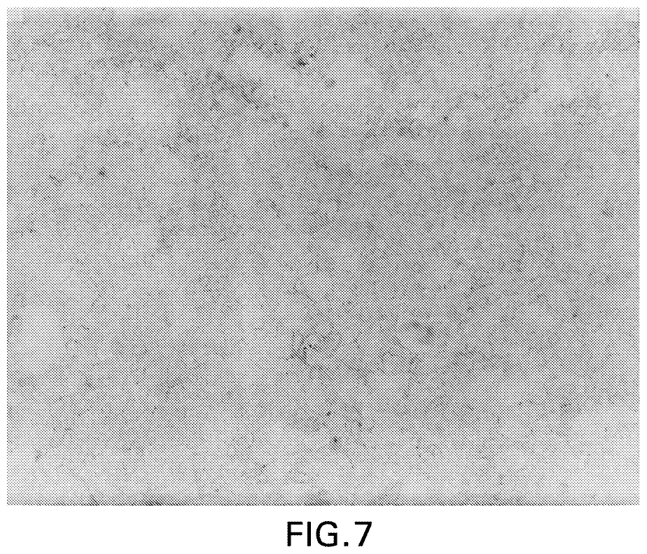

Approximately 800 fluorescent images were stitched together in a representative image to demonstrate extensive cellular coverage on the membrane. FIG. 7 is a representative image of one of the stitched images in Example 3 that shows the cobblestone-like morphology of individual cells growing on a scaffold, similar to those published in A microfluidics-based in vitro model of the gastrointestinal human-microbe interface, (Shah et al., Nat Commun 7:11535 (2016) doi: 10.1038/ncomms11535).

### Example 4: Secreted Products in Host-Microbe Co-Culture Induce V. Harveyi QS

A previous report has demonstrated that indirect co-culture (i.e. via secreted interaction) of Vibrio harveyi with human epithelial cells induces human-cell production of an effector molecule that induces bioluminescence in a V. harveyi reporter strain (Cell Host & Microbe 19, 470-480 (2016)). This communication axis was recapitulated in the bioreactor, with V. harveyi MM32 (luxN::Cm, luxS::Tn5Kan) in the membrane inside Chamber 1 and Caco-2 cells grown on a membrane in Chamber 2.

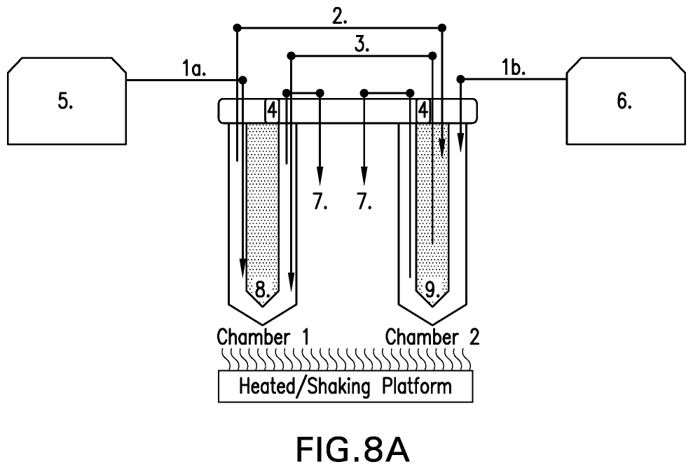

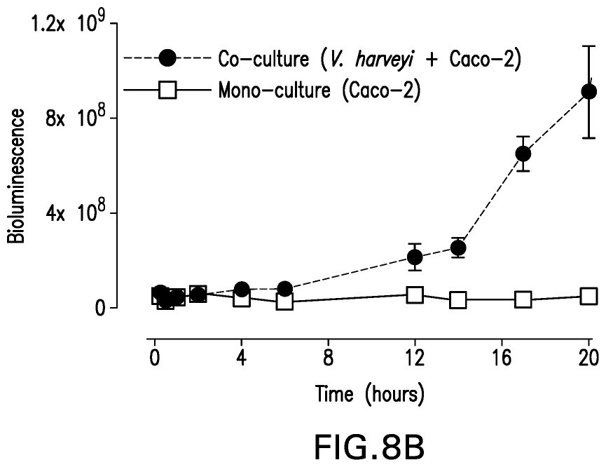

This experiment comprised a series of bioreactors using the setup as shown in FIG. 8A.

Membranes (1000 kD, Spectrum Labs, G235062) were completely immersed in water for ˜1 min to wash. The membranes were removed, the excess water was poured off, and the membranes were completely immersed in molecular biology grade 200 proof ethanol (Fisher Scientific) for 1 min. The membranes were removed, EtOH was removed by aspiration and the membranes were allowed to air dry for 30 min in a biosafety cabinet. The inside of applicable membranes were treated with 150 ug/ml collagen (Gibco, A1048301) in sterile PBS with 20 mM acetic acid (Sigma). The membranes were placed in a 37° C. incubator for 3 h. The membranes were removed, collagen solutions were aspirated, and the membranes were washed with sterile PBS. The collagen-coated membranes in chamber 2 were stored in 50 ml centrifuge tubes at 4° C. until use. The inside of membrane in chamber 2 was seeded with 5 ml Caco-2 cells (5e5 cells/ml) in DMEM with 10% FBS and penicillin/streptomycin. The outside of the membrane (in a 50 ml centrifuge tube) was filled with ˜33 ml of the same media. The inside of the membrane was sealed using a threaded cap to prevent cells from leaking out. Tubes were placed on a tube roller in a 37° C., 5% CO₂ incubator and the tube roller was initially set at 30 rpm. Rotational speed was steadily reduced at ˜1 rpm/sec until 0.5 rpm was reached. The tubes were left rolling for 24 h. After 24 h, the tubes were placed upright in a 37° C. 5% CO₂ incubator, and the caps were loosened to allow air flow. Three days after the cells were seeded, the media was aspirated from the outside of the membrane and replaced with fresh media.

One day prior to setting up the bioreactor systems, the bioreactor systems were sterilized by autoclaving, and a small swab of frozen Vibrio harveyi MM32 (ATCC BAA-1121) in 30% glycerol was used to inoculate 5 ml of marine medium (Boston Bioproducts) and was incubated overnight at 30° C.

FIG. 8A is a schematic diagram representing a dual chamber bioreactor system as described in Example 4. The bioreactor comprises two chambers, wherein each bioreactor system has an EtOH-sterilized membrane attached inside chamber 1. Autoinducer bioassay (AB) media is supplied to chamber 1 where Dulbecco's Modified Eagle Medium (DMEM) is supplied to chamber 2. Arrowed lines on schematic are representative of the fluidic tubing (and flow direction) on the apparatus and are not drawn to scale. The following numbers on FIG. 8A are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Cell media into chamber 1 (AB Media).
6. Cell media into chamber 2 (DMEM).
7. Outputs for sampling.
8. Cell culture (V. harveyi or blank).
9. Cell culture (Caco-2 cells).

Seven days after seeding epithelial cells on applicable membranes, the bioreactor systems were assembled as shown in FIG. 8A, each with two chambers, each bioreactor system having an EtOH-sterilized membrane attached inside chamber 1. The chamber 1 membrane was filled with 4 ml of AB medium immediately before attaching to bioreactor system. The membrane with Caco-2 cells adhered was attached to the bioreactor system in chamber 2. AB media was connected to input pumps for all chamber is and DMEM (with 10% FBS, no antibiotics) was connected to input pumps for all chamber 2s. Media was pumped into each chamber outside the membrane until entire membrane was covered (˜33 ml/chamber). Fluidics pumps were initiated at flow rates of 0.1 ml/min into and out of each chamber and 0.25 ml/min back-and-forth between the outside of the membrane in chamber 1 and inside of membrane in chamber 2. Shaking was set at 150 rpm and the temperature was set to 30° C. 1 ml of the overnight culture of V. harveyi MM32 was injected with needled syringe through the rubber septum into the membrane of chamber 1. Samples were collected on 96-well deep well plates via fraction collector at predetermined timepoints. The plates with samples were stored at −80° C. until use in downstream bioassay.

A small swab of frozen Vibrio harveyi MM32 (ATCC BAA-1121) in 30% glycerol was used to inoculate 5 ml of marine medium (Boston Bioproducts) and was incubated overnight at 30° C.

V. harveyi MM32 overnight culture was diluted 1:1000 in Autoinducer bioassay (AB) medium (Boston Bioproducts) with 0.1 mM boric acid (Sigma) to make a working solution. Samples collected from the bioreactor (both V. harveyi/Caco-2 co-culture and Caco-2 mono-culture control) were mixed with the working solution 4:6 (40% v/v). Samples were distributed on a 384 well plate in triplicate. The plate was placed in a Biotek Synergy Neo2 multi-mode reader set at 30° C. Every half hour, the plate was shaken for 10 sec and then absorbance (600 nm) and luminescence were measured.

FIG. 8B is a line graph showing the bioluminescence data of time-dependent supernatants from bioreactor in V. harveyi MM32 bioassay. FIG. 8B shows a clear difference in bioluminescence induction between the co-culture and the monoculture, indicating that the mammalian cells produced a signal in response to the bacteria present. As shown in FIG. 8B, data displayed is bioluminescence signal normalized to reporter bacterial growth (A600) at the 8 h timepoint.

There is a clear difference in bioluminescence induction between the co-culture and the monoculture. The monoculture tracks at background level bioluminescence through 20 h. Contrarily, the co-culture supernatant steadily increases bioluminescence above background levels throughout the experiment, indicating that the mammalian cells produced a signal in response to the bacteria present. The first 2-6 h of experimental set up showed similar levels of bioluminescence induction between the monoculture and the coculture, indicating that sufficient bacterial signal was not yet received or sufficient mammalian signal was not yet produced.

### Example 5: Establishing Anoxic Conditions in an Individual Chamber while Maintaining Aerobic Conditions in Separate Chamber (Integrated Anaerobic Media Input)

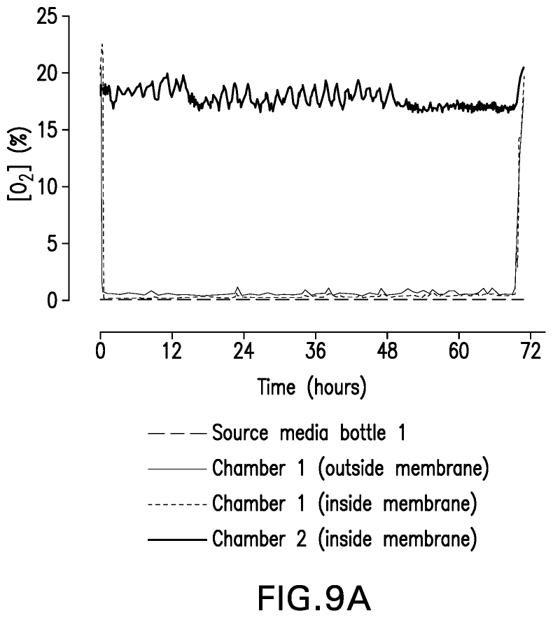

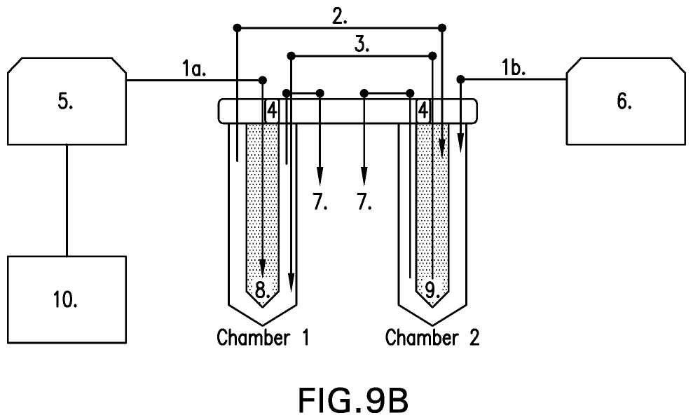

FIG. 9B is a schematic diagram representing the dual chamber bioreactor system as described in Example 5. The bioreactor comprises the tubing connected from a house nitrogen line to one PBS bottle (Source media bottle 1). Arrowed lines on schematic are representative of the fluidic tubing (and flow direction) on the apparatus and are not drawn to scale Chamber 1 is in an anaerobic environment. Chamber 2 is an aerobic environment. The following numbers on FIG. 9B are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Source/cell media into chamber 1 (PBS).
6. Source/cell media into chamber 2 (PBS).
7. Outputs for sampling.
8. Distinct cell culture or blank.
9. Distinct cell culture or blank.
10. Anaerobic gas mixture (nitrogen).
   The bioreactor system shown in FIG. 9B comprises two chambers. Two 2-liter bottles of PBS were used to supply the bioreactor systems. Tubing connected a house nitrogen line to one PBS bottle. A port at the top of the bottle was used to release effluent gas. This port was connected via tubing to chamber 1, outside the membrane. When nitrogen was turned on, the input PBS bottle was purged, followed by chamber 1. As shown in FIG. 9B, the bioreactor system was assembled with two chambers, each with a membrane attached inside. The chamber 1 membrane was filled with 4 ml of PBS immediately before attaching to the bioreactor system. The PBS media was pumped into each chamber outside the membrane until the entire membrane was covered (˜33 ml/chamber). All other fluidics pumps and tubing (inputs, outputs, and mixing) were connected. Three oxygen microsensors (PreSens) were inserted into various locations within the bioreactor system: (i) chamber 1, outside the membrane; (ii) chamber 1, inside the membrane, and (iii) chamber 2, inside the membrane. There was also an oxygen dipping probe (PreSens) placed in source media bottle 1. Nitrogen gas was released into system, oxygen concentration measurements were started, and fluidic pumps were turned on so that PBS was being added and removed at 0.1 ml/min in each chamber. Two fluidic pumps were used to circulate the PBS from outside the membrane in chamber 1 to inside the membrane in chamber 2 (and reverse direction) at 0.25 ml/min. Immediately after starting the system, 55 μl EC-Oxyrase was injected into the inside of the membrane in chamber 1. Oxygen concentration measurements were recorded for approximately 3 days and are shown in FIG. 9A. FIG. 9A is a line graph showing oxygen concentration measurements for approximately 3 days in 3 locations within the bioreactor system: (i) Chamber 1, outside the membrane; (ii) Chamber 1, inside the membrane, and (iii) Chamber 2, inside the membrane, and additionally an oxygen dipping probe placed in source media bottle 1. For clarity, Source media bottle 1, as shown in the graph is at constant O₂ level of zero percent. The chamber 2 (inside membrane) O₂ level varies but is represented at just below 20 percent.

Oxygen levels in chamber 1 dropped significantly upon nitrogen purging and addition of EC-Oxyrase. Oxygen levels stayed consistently below 0.5% v/v for a period of nearly 3 days (chamber 1, inside and outside membrane). Around 72 h, the sensors were removed as the bioreactor system was disassembled and sensors recorded typical atmospheric oxygen levels. The oxygen level inside the membrane in chamber 2 (where mammalian cells might be located in some embodiments) remained between 15-20% throughout the duration of the experiment, demonstrating an oxygenated environment.

### Example 6: Inoculating the Bioreactor System with Anaerobic Bacteria and Monitoring Growth Over Time. Example 6 Comprises Two Separate Experiments with Fusobacterium nucleatum and Faecalibacterium prausnitzii

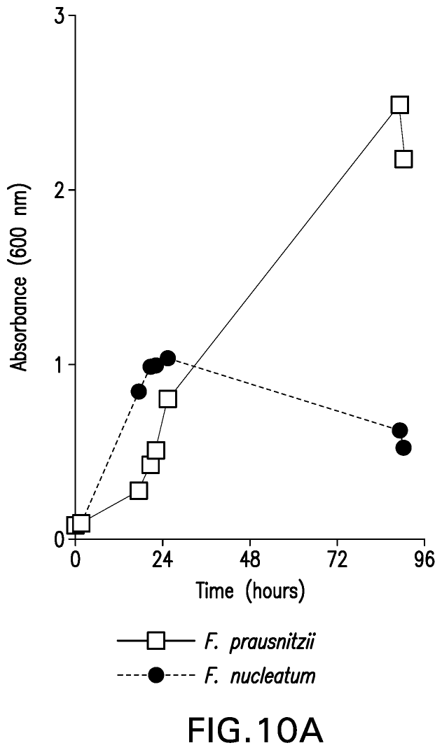

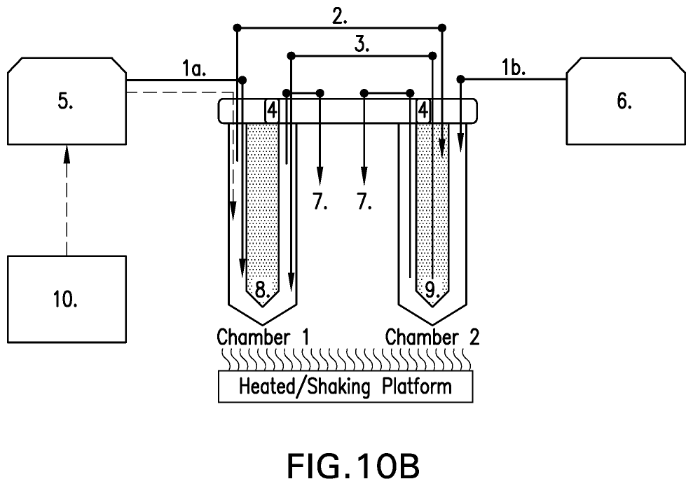

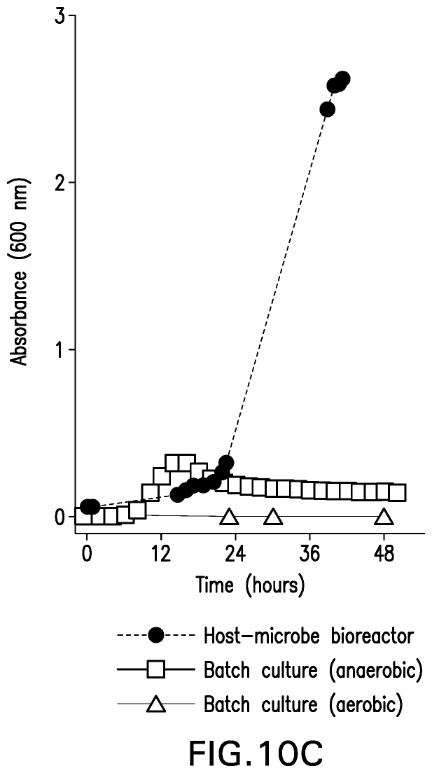

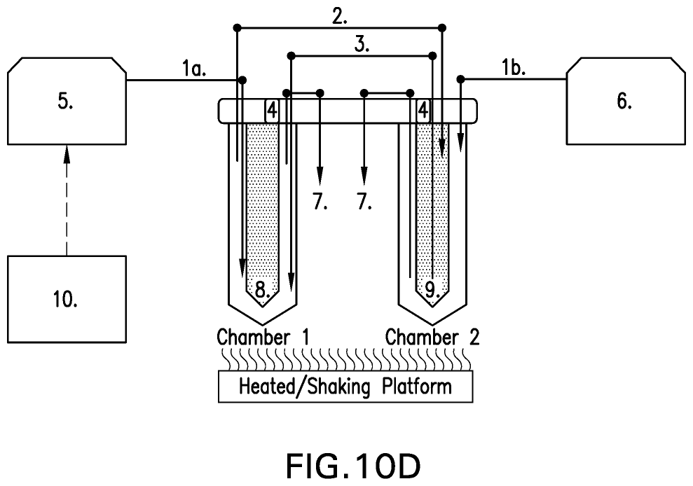

Before initiating an experiment for each cell type, Fusobacterium nucleatum VPI 4351 (ATCC 23726) and Faecalibacterium prausnitzii A2-165 (DSMZ17677) were separately streaked onto BHI agar and YCFAC agar plates (Anaerobe Systems), respectively, and incubated at 37° C. in an anaerobic chamber. 24 h prior to the start of the experiment, individual colonies were picked and used to inoculate 5 ml of YCFAC broth (Anaerobe Systems), which were individually incubated at 37° C. in an anaerobic chamber. FIG. 10A is a line graph representing the density of Fusobacterium nucleatum and Faecalibacterium prausnitzii, as examples of anaerobic bacteria, as used in Example 6. FIG. 10B is a schematic diagram representing the dual chamber bioreactor system as described in Example 6. The bioreactor comprises Yeast Casitone Fatty Acids Broth with Carbohydrates (YCFAC) media is supplied to chamber 1 and PBS is supplied to chamber 2. The bioreactor additionally comprises an anaerobic gas mixture connected to the YCFAC bottle. Arrowed lines on schematic are representative of the fluidic tubing (and flow direction) on the apparatus and are not drawn to scale The dashed line indicates the flow of gas. The following numbers on FIG. 10B are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Source/cell media into chamber 1 (YCFAC).
6. Source/cell media into chamber 2 (PBS).
7. Outputs for sampling.
8. Cell culture (Bacterial—F. prausnitzii or F. nulceatum).
9. Separate cell culture.
10. Anaerobic gas mixture (nitrogen).

The bioreactor system, as shown in FIG. 10B, was sterilized by autoclaving. YCFAC broth comprised the media bottle supplying chamber 1. PBS comprised the media bottle supplying chamber 2. Anaerobic gas mix (Middlesex Gases & Technologies) is connected to the YCFAC bottle via tubing and in-line pressure regulator (Omega). As shown in FIG. 10B, a port at the top of the bottle was used to release effluent gas. This port was connected via tubing to chamber 1, outside the membrane. When anaerobic gas is turned on, the input media bottle to chamber 1 is purged, followed by chamber 1. The bioreactor system was assembled with two chambers, each with an EtOH-sterilized membrane attached inside. The chamber 1 membrane was filled with 4 ml of YCFAC immediately before attaching to bioreactor system. YCFAC media was pumped into chamber 1 outside the membrane and PBS was pumped into chamber 2 outside the membrane until entire membrane was covered (˜33 ml/chamber).

Fluidics pumps were initiated at flow rates of 0.1 ml/min into and out of each chamber and 0.25 ml/min back-and-forth between the outside of the membrane in chamber 1 and inside of the membrane in chamber 2. Shaking was set at 250 rpm, and the temperature was set to 37° C. Immediately after starting the system, 55 μl EC-Oxyrase was injected into the inside of the membrane in chamber 1. The overnight cultures of F. nucleatum and F. prausnitzii (50 μl) were transferred independently from the anaerobic chamber to the inside of the membrane in chamber 1 via a needled syringe and septum port (into separate bioreactor systems). At various timepoints samples were removed from inside of the membrane in chamber 1 using a needled syringe, 100 μl was aliquoted onto a clear 96-well plate, and absorbance at 600 nm was measured using a PHERAstar FSX multi-mode microplate reader.

As shown in FIG. 10A, over the course of the experiment (˜4 days), the growth of both bacterial species, F. nucleatum and F. prausnitzii, increased inside the membrane of chamber 1 in separate systems, indicating bacterial growth within the bioreactor system. Much of this growth can be observed within the first 24 h.

### Comparing Growth of an Anaerobic Bacterium in the Bioreactor Vs Growth in Batch Culture (Both Anaerobic and Aerobic).

Before initiating the experiment, Fusobacterium nucleatum VPI 4351 [1210] (ATCC 23726) was streaked onto BHI agar (Anaerobe Systems) and incubated at 37° C. in an anaerobic chamber. Three days prior to the start of the experiment, an individual colony was picked and used to inoculate 5 ml of BHI broth (also referred to as BHI or BHI Media) (Anaerobe Systems), which was incubated at 37° C. in an anaerobic chamber. The bioreactor system was sterilized by autoclaving. BHI broth comprised the media bottle supplying chamber 1. PBS comprised the media bottle supplying chamber 2. Anaerobic gas mix (Middlesex Gases & Technologies) was connected to the BHI bottle via tubing and in-line pressure regulator (Omega). A port at the top of the bottle was used to release effluent gas. FIG. 10D is a schematic diagram representing the dual chamber bioreactor system as described in Example 6. The dual chamber system comprises brain heart infusion (BHI) media pumped into chamber 1 outside the membrane and PBS pumped into chamber 2 outside the membrane. The bioreactor additionally comprises an anaerobic gas mixture connected to the BHI media bottle. Arrowed lines on schematic are representative of the fluidic tubing (and flow direction) on the apparatus and are not drawn to scale The dashed line indicates the flow of gas. The following numbers on FIG. 10D are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Source/cell media into chamber 1 (BHI Media).
6. Source/cell media into chamber 2 (PBS).
7. Outputs for sampling.
8. Cell culture (bacterial—F. nulceatum).
9. Separate cell culture.
10. Anaerobic gas mixture.

As shown in FIG. 10D, this port was connected via tubing to chamber 1, outside the membrane. When anaerobic gas was turned on, the input media bottle to chamber 1 was purged, followed by chamber 1. The bioreactor system was assembled with two chambers, each with an EtOH-sterilized membrane attached inside. The chamber 1 membrane was filled with 4 ml of BHI immediately before attaching to bioreactor system. BHI media was pumped into chamber 1 outside the membrane and PBS was pumped into chamber 2 outside the membrane until entire membrane was covered (˜33 ml/chamber). Fluidics pumps were initiated at flow rates of 0.1 ml/min into and out of each chamber and 0.25 ml/min back-and-forth between the outside of the membrane in chamber 1 and inside of membrane in chamber 2. Shaking was set at 250 rpm, and the temperature was set to 37° C. Immediately after starting the system, 55 μl EC-Oxyrase was injected into the inside of the membrane in chamber 1. The broth culture of F. nucleatum (100 μl) was transferred from the anaerobic chamber to the inside of the membrane in chamber 1 via a needled syringe and septum port (into separate bioreactor systems). At various timepoints, samples were removed from inside of the membrane in chamber 1 using a needled syringe, 100 μl was aliquoted onto a clear 96-well plate, and absorbance at 600 nm was measured using a PHERAstar FSX multi-mode microplate reader.

For batch culture experiments, in an anaerobic chamber, 48 h broth culture of F. nucleatum was diluted 1:50 into BHI broth. Aliquots of 100 μl were distributed amongst two clear 96-well plates. One plate was placed in a Biotek Epoch2 microplate reader set at 37° C. inside the anaerobic chamber. The plate was shaken every 2 h and absorbance measured at 600 nm. The other plate was removed from the anaerobic chamber and placed in a 37° C., 5% CO₂ incubator. At intermittent timepoints, the plate was removed and absorbance at 600 nm was measured in a Biotek Neo2 multi-mode microplate reader. Both plates were measured over the course of approximately 48 h.

The anaerobic batch culture displayed typical in vitro bacterial growth behavior in a rich medium including lag phase early on, logarithmic growth, and then stationary phase, with absorbance values never exceeding 0.4. Contrarily, the bacteria grown in the perfusion bioreactor system showed growth to substantially higher densities, reaching absorbance values of ˜2.6. This indicates that this bioreactor system allows for bacterial growth at much higher densities than standard in vitro batch culture. As expected, bacteria grown in an aerobic environment did not display any growth.

FIG. 10C is a line graph showing the results of Example 6. The anaerobic batch culture displayed typical in vitro bacterial growth behavior in a rich medium including lag phase early on, logarithmic growth, and then stationary phase, with absorbance vales never exceeding 0.4. The host-microbe bioreactor (the bacteria grown in the perfusion bioreactor) showed growth to substantially higher densities. As expected, bacteria grown in aerobic environment did not display any growth.

### Example 7: Growth of Anaerobic Bacteria in Co-Culture with Caco-2 Cells (Human Epithelial) in Bioreactor System

Example 7 comprised a series of bioreactor systems.

Caco-2 cells grow in a monolayer on a membrane and display epithelial barrier function. Membranes (1000 kD, Spectrum Labs, G235062) were completely immersed in water for ˜1 min to wash. Membranes were removed, the excess water was poured off, and the membranes completely immersed in molecular biology grade 200 proof ethanol (Fisher Scientific) for 1 min. The membranes were removed, EtOH was removed by aspiration and the membranes were allowed to air dry for 30 min in a biosafety cabinet. When applicable, the inside of the membranes comprising Caco-2 cells were treated with 150 ug/ml collagen (Gibco, A1048301) in sterile PBS with 20 mM acetic acid (Sigma). The membranes were placed in a 37° C. incubator for 3 h. The membranes were removed, collagen solutions were aspirated, and the membranes were washed with sterile PBS. The collagen-coated membranes were stored in 50 ml centrifuge tubes at 4° C. until use. The inside of membranes were seeded with 5 ml Caco-2 cells (1e6 cells/ml) in DMEM with 10% FBS and penicillin/streptomycin.

The outside sections of the membrane (in 50 ml centrifuge tube) were filled with ˜33 ml of the same media. The inside sections of the membranes were sealed using a threaded cap to prevent cells from leaking out. Tubes were placed on a tube roller in a 37° C., 5% CO₂ incubator and the tube roller was initially set at 30 rpm. Rotational speed was steadily reduced at ˜1 rpm/sec until 0.5 rpm was reached. The tubes were left rolling for 24 h. After 24 h, the tubes were placed upright in a 37° C. 5% CO₂ incubator, and the caps were loosened to allow air flow. Three days after the cells were seeded, the media was aspirated from the outside of the membrane and replaced with fresh media. Seven days after cells were seeded, media was removed by aspiration from all tubes (inside and outside of membrane) and used for an experiment in the bioreactor system.

Before initiating the separate experiments, Fusobacterium nucleatum VPI 4351 [1210] (ATCC 23726) and Faecalibacterium prausnitzii A2-165 (DSMZ17677) were individually streaked onto BHI agar and YCFAC agar plates (Anaerobe Systems), respectively, and the plates were incubated at 37° C. in an anaerobic chamber. 24 h prior to the start of the experiment, individual colonies were picked and used to inoculate 5 ml of YCFAC broth (Anaerobe Systems), which were incubated at 37° C. in an anaerobic chamber.

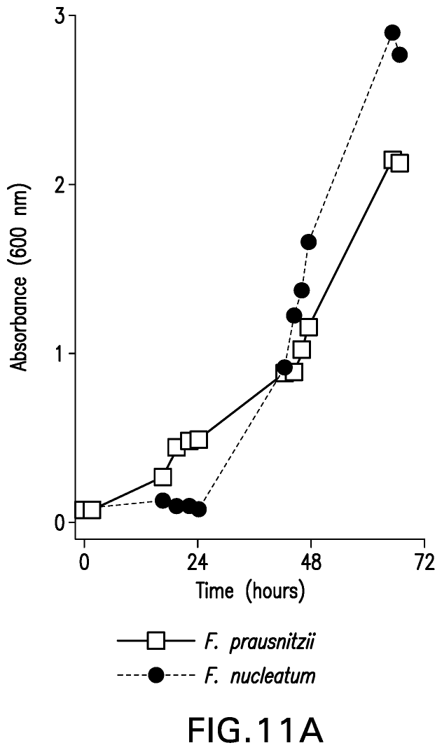

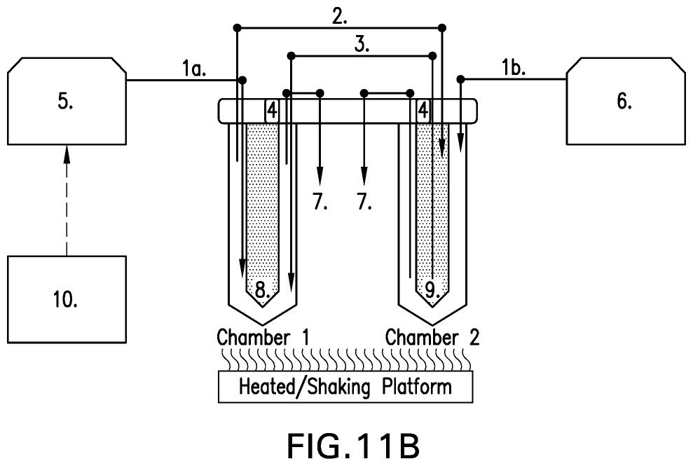

The bioreactor system was sterilized by autoclaving. FIG. 11B is a schematic diagram representing the dual chamber bioreactor system as described in Example 7. The dual-chamber bioreactor comprises YCFAC broth supplied to chamber 1 and DMEM supplied to chamber 2. Additionally, the bioreactor comprises an anaerobic gas mixture connected to the YCFAC bottle. Chamber 1 comprises bacterial cells. Chamber 2 comprises epithelial cells (Caco-2 cells) with a collagen coated membrane. Arrowed lines on schematic are representative of the fluidic tubing (and flow direction) on the apparatus and are not drawn to scale The dashed line indicates the flow of gas. The following numbers on FIG. 11B are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Cell media source into chamber 1 (YCFAC).
6. Cell media source into chamber 2 (DMEM).
7. Outputs for sampling.
8. Cell culture (Bacterial: F. prausnitzii or F. nulceatum).
9. Cell culture (Epithelial cells: Caco-2 cells inside the membrane of chamber 2).
10. Anaerobic gas mixture.

As shown in FIG. 11B, YCFAC broth comprised the media bottle supplying chamber 1. DMEM with 10% FBS (v/v) comprised the media bottle supplying chamber 2. Anaerobic gas mix (Middlesex Gases & Technologies) was connected to the YCFAC bottle via tubing and an in-line pressure regulator (Omega). A port on the top of the bottle was used to release effluent gas. This port was connected via tubing to chamber 1, outside the membrane. When anaerobic gas was turned on, the input media bottle to chamber 1 was purged, followed by chamber 1. Chamber 1 had an EtOH-sterilized membrane attached inside. Chamber 2 had an epithelial layer comprising caco-2 cells on the inner membrane. The chamber 1 membrane was filled with 4 ml of YCFAC immediately before attaching to bioreactor system. YCFAC media was pumped into chamber 1 outside the membrane and DMEM with 10% FBS was pumped into chamber 2 outside the membrane until the entire membrane was covered (˜33 ml/chamber). Fluidics pumps were initiated at flow rates of 0.1 ml/min into and out of each chamber and 0.25 ml/min back-and-forth between the outside of the membrane in chamber 1 and inside of membrane in chamber 2. Shaking was set at 250 rpm, and the temperature was set to 37° C. Immediately after starting the system, 55 μl EC-Oxyrase was injected into the inside of the membrane in chamber 1. The overnight cultures of F. nucleatum and F. prausnitzii (50 μl) were individually transferred from the anaerobic chamber to the inside of the membrane in chamber 1 via a needled syringe and septum port (into separate bioreactor systems). At various timepoints samples were removed from inside of the membrane in chamber 1 using a needled syringe, 100 μl was aliquoted onto a clear 96-well plate, and absorbance at 600 nm was measured using a PHERAstar FSX multi-mode microplate reader.

FIG. 11A is a line graph showing the results of Example 7, where both bacterial species are able to grow in the bioreactor system in anaerobic YCFAC media when co-cultured with Caco-2 cells and DMEM with 10% Fetal Bovine Serum (FBS). As shown in FIG. 11A, both bacterial species were able to grow in the bioreactor system in anaerobic YCFAC media when co-cultured with Caco-2 cells and DMEM with 10% FBS. As observed in other experiments, bacterial growth reached extremely high cell densities (A₆₀₀=2-3), indicating that this environment promotes substantial bacterial growth.

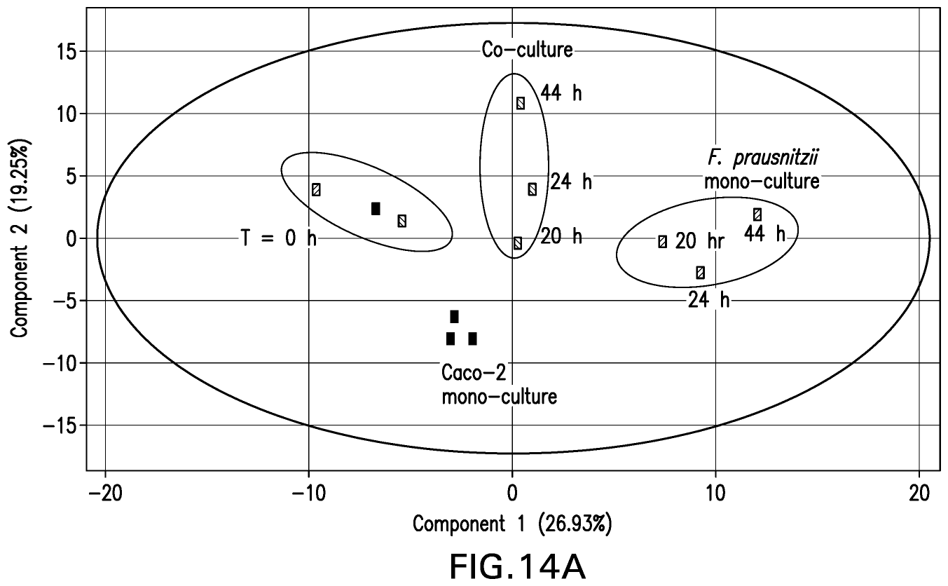

Supernatants from the outside the membrane in chamber 1 were collected at multiple timepoints were and 200 known metabolites were measured using an Agilent 6470 Triple Quadrupole (QQQ) mass spectrometer, coupled to an Agilent 1290 Infinity II HPLC with quaternary pump was used. Data was analyzed using Agilent MassHunter Quantitative Analysis B.08 software. FIG. 14A is a scatter plot summarizing the Principal Component Analysis (PCA) of 200 metabolites measured by mass spectrometry (MS) when F. prausnitzii and Caco-2 cells are co-cultured, as described in Example 7. As shown in FIG. 14A, temporal changes in metabolites levels can be seen. Additionally, shown in FIG. 14A, the metabolomic environment is different in a host-microbe co-culture arrangement in comparison to mono-cultures of either species.

### Example 8: Additional Arrangements of the Bioreactor System Due to Designed Flexibility

Because of its designed flexibility, there are other arrangements with which the bioreactor system could have added utility

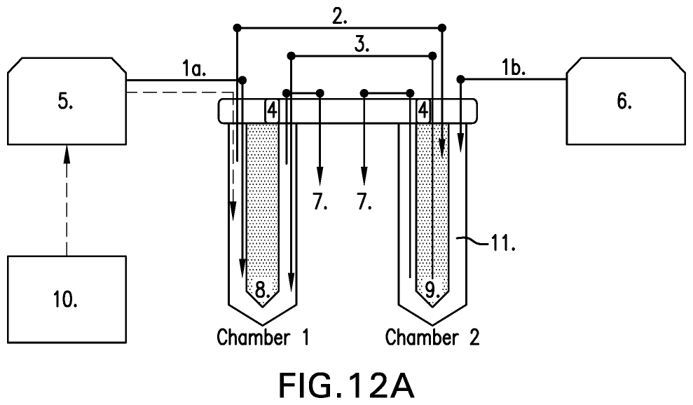

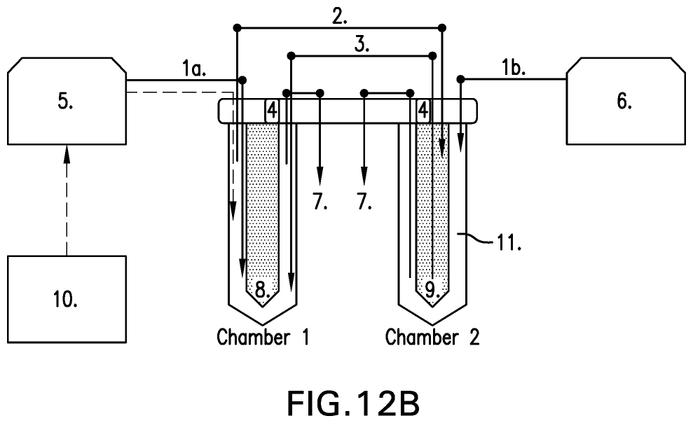

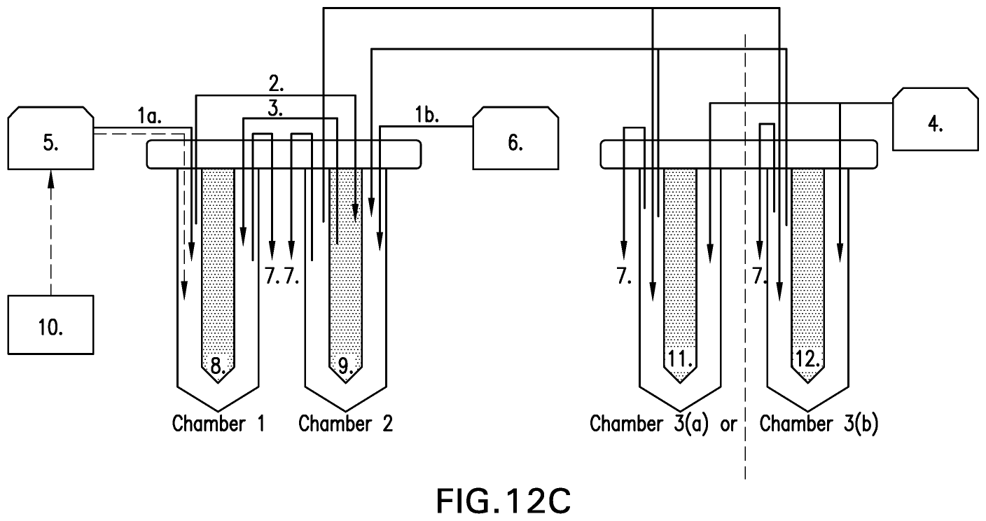

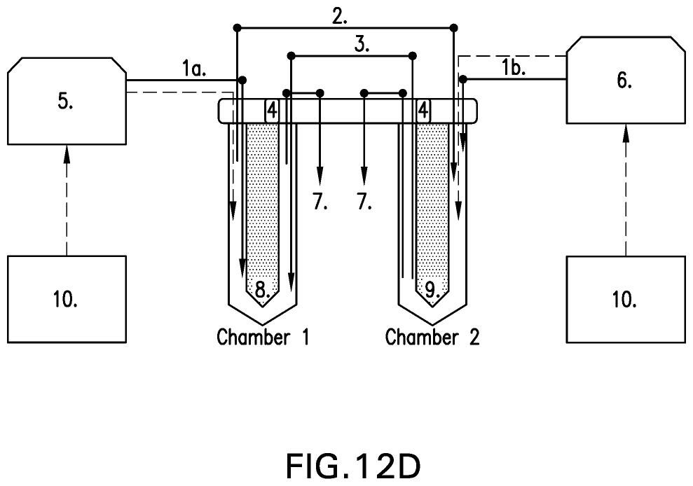

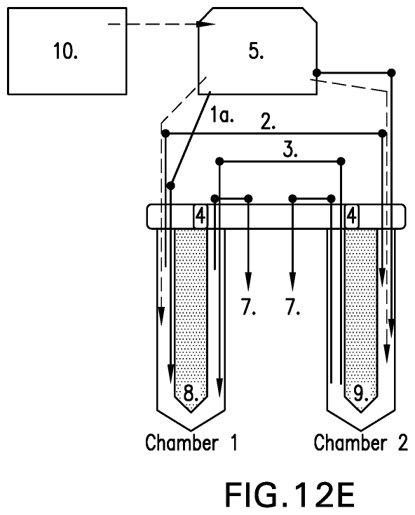

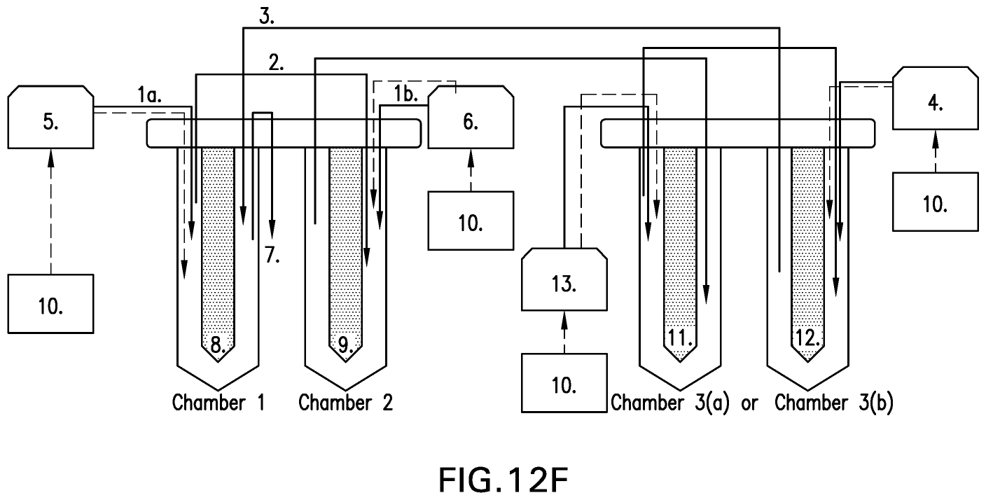

Immune cells could be added to the basal side of the epithelial cells on the membrane (analogous to in vivo arrangement) as represented in FIG. 12A. FIG. 12A is a schematic diagram representing a dual chamber bioreactor system as described in Example 8. The bioreactor comprises immune cells added to the basal side (outside), shown by ‘11’ of the epithelial cells on the membrane (analogous to in vivo arrangements). Arrowed lines on schematic are representative of the fluidic tubing (and flow direction) on the apparatus and are not drawn to scale The dashed line indicates the flow of gas. The following numbers on FIG. 12A are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Cell media source into chamber 1 (Source Media Bottle).
6. Cell media source into chamber 2 (Source Media Bottle).
7. Outputs for sampling.
8. Cell culture (Bacterial cells).
9. Cell culture inside the membrane in chamber 2 (Epithelial cells).
10. Anaerobic gas mixture.
11. Cell culture outside the membrane (basal side) in chamber 2 (Immune cells).

Immune cells can be added without epithelial cells, allowing for more simple co-culture between bacteria and immune cells as represented in FIG. 12B. FIG. 12B is a schematic diagram representing the dual chamber bioreactor system as described in Example 8. The bioreactor comprises immune cells added without epithelial cells, allowing for more simple co-culture between bacteria and immune cells. Arrowed lines on schematic are representative of the fluidic tubing (and flow direction) on the apparatus and are not drawn to scale The dashed line indicates the flow of gas. The following numbers on FIG. 12B are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Septum port/valve (orifice).
5. Cell media source into chamber 1 (Source Media Bottle).
6. Cell media source into chamber 2 (Source Media Bottle).
7. Outputs for sampling.
8. Cell culture (Bacterial cells)
9. Cell culture or control with no cells.
10. Anaerobic gas mixture
11. Immune cells.

As represented in 12C, immune cells (or cell types representing different organs) can be added in additional bioreactor systems, in the membrane or outside the membrane. In the examples, it might be useful to have cells added in the membrane to keep them in place throughout experiment. Adding cells outside the membrane would allow automated sampling of the cells over time. Automated sampling can also be done from inside them membrane. FIG. 12C is a schematic diagram representing a dual chamber bioreactor system as described in Example 8. The bioreactor comprises a setup where immune cells (or cell types representing different organs) are added to additional bioreactor systems, in the membrane or outside the membrane. Arrowed lines on schematic are representative of the fluidic tubing (and flow direction) on the apparatus and are not drawn to scale The dashed line indicates the flow of gas. The following numbers on FIG. 12C are identified as the following:

1a. Media input into chamber 1.

1b. Media input into chamber 2.

2. Exchange of contents from chamber 1 into chamber 2 through a hollow conduit.

3. Exchange of contents from chamber 2 into chamber 1 through a hollow conduit.

5. Cell media source into chamber 3 (Source Media Bottle).

5. Cell media source into chamber 1 (Source Media Bottle).

6. Cell media source into chamber 2 (Source Media Bottle).

7. Outputs for sampling.

8. Cell culture (Bacterial cells).

9. Cell culture (Epithelial cells).

10. Anaerobic gas mixture

11. Cell culture (Immune cells).

12. Cell culture (Immune cells).

As shown in FIG. 12D, multiple bacteria can be indirectly co-cultured in the bioreactor system (with no human cells). They can have different media inputs that reflect favorable conditions for each bacterial type. FIG. 12D is a schematic diagram representing a dual chamber bioreactor system, wherein multiple bacteria can be indirectly co-cultured in the bioreactor system with no human cells. The different media inputs reflect favorable conditions for each bacterial type. Arrowed lines on schematic are representative of the fluidic tubing (and flow direction) on the apparatus and are not drawn to scale The dashed line indicates the flow of gas. The following numbers on FIG. 12D are identified as the following:

1a. Media input into chamber 1.

1b. Media input into chamber 2.

2. Exchange of contents from chamber 1 into chamber 2 through a hollow conduit.

3. Exchange of contents from chamber 2 into chamber 1 through a hollow conduit.

4. Septum port/valve (orifice).

5. Cell media into chamber 1 (Bacterial media).

6. Cell media into chamber 2 (Bacterial media).

7. Outputs for sampling.

8. Cell culture (Bacterium 1).

9. Cell culture (Bacterium 2).

10. Anaerobic gas mixture.

As shown in FIG. 12E, multiple bacteria can be indirectly co-cultured in the bioreactor system (with no human cells). They can have the same media inputs. FIG. 12E is a schematic diagram representing a dual chamber bioreactor system, wherein multiple bacteria can be indirectly co-cultured in the bioreactor system with no human cells and have the same media inputs. Arrowed lines on schematic are representative of the fluidic tubing (and flow direction) on the apparatus and are not drawn to scale The dashed line indicates the flow of gas. The following numbers on FIG. 12E are identified as the following:
1a. Media input into chamber 1.
2. Exchange of contents from chamber 1 into chamber 2 through a hollow conduit.
3. Exchange of contents from chamber 2 into chamber 1 through a hollow conduit.
4. Septum port/valve (orifice).
5. Cell media into chamber 1 and 2 (Bacterial media).
7. Outputs for sampling.
8. Cell culture (Bacterium 1).
9. Cell culture (Bacterium 2).
10. Anaerobic gas mixture.

As shown in FIG. 12F, more than two bacteria/chambers can be co-cultured in the bioreactor system. FIG. 12F is a schematic diagram representing four bacteria chambers co-cultured in the bioreactor system. Arrowed lines on schematic are representative of the fluidic tubing (and flow direction) on the apparatus and are not drawn to scale The dashed line indicates the flow of gas. Sampling the contents from outside the membrane is shown only for the first chamber but is applicable to all chambers (represented by number 7). The following numbers on FIG. 12F are identified as the following:
1a. Media input into chamber 1.
1b. Media input into chamber 2.
2. Exchange of contents from chamber 1 into chamber 2 through a hollow conduit.
3. Exchange of contents from chamber 2 into chamber 1 through a hollow conduit.
4. Cell media into chamber n (Bacterial media n).
5. Cell media into chamber 1 (Bacterial media 1).
6. Cell media into chamber 2 (Bacterial media 2).
7. Outputs for sampling.
8. Cell culture (Bacterium 1).
9. Cell culture (Bacterium 2).
10. Anaerobic gas mixture
11. Cell culture (Bacterium 3).
12. Cell culture (Bacterium n).
13. Source/cell media into chamber 3 (Bacterial Media 3).

This bioreactor system comprises off the shelf parts structured in a way to enable communication between environments in a bioreactor system.

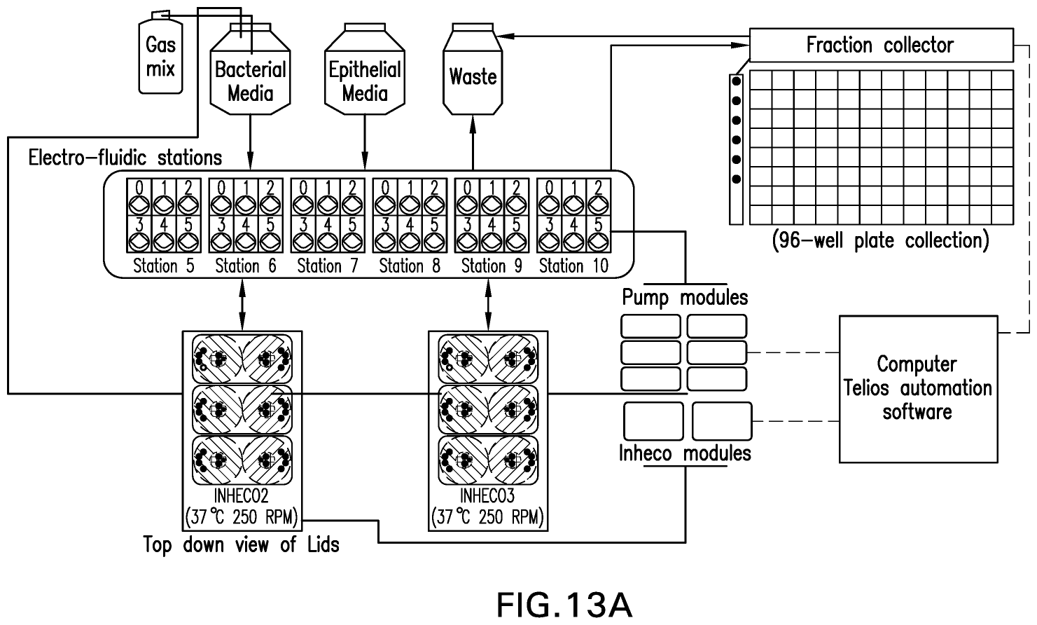

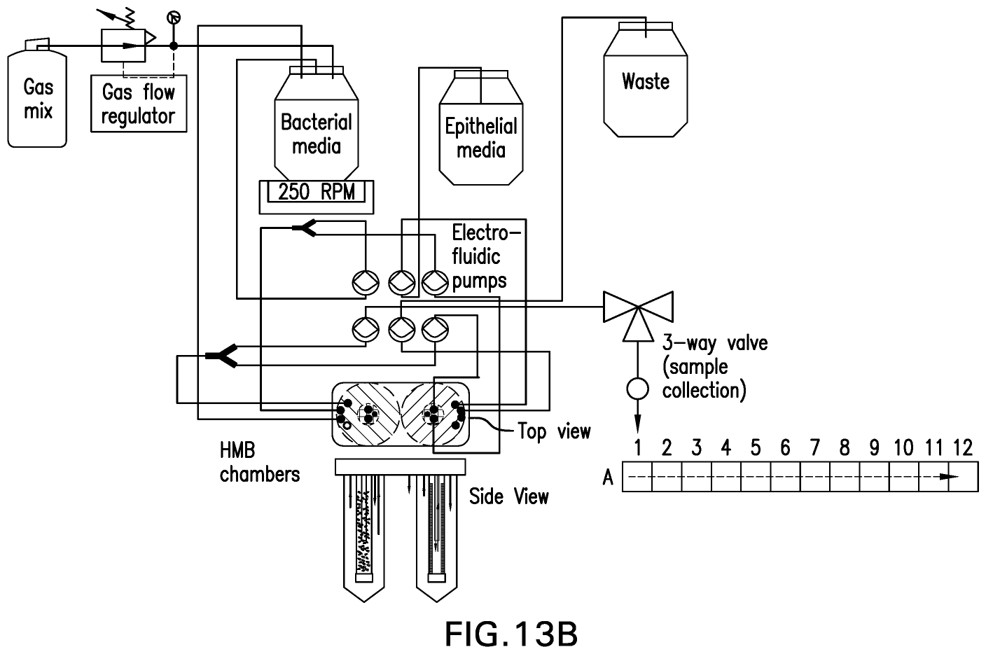

Due to the modular approach to the design of this bioreactor system, the bioreactor system can be scaled according to the desired number of cell chambers and modules as represented in FIG. 13A and FIG. 13B. FIG. 13A is a schematic diagram of one potential configuration of the bioreactor. FIG. 13A comprises 5 modules, each comprising 3 bioreactor systems (3 dual chamber systems) each comprising their own pump station communicating to the computer. FIG. 13B is a schematic diagram of one potential configuration of the bioreactor.
FIG. 13B comprises 1 module or bioreactor system comprising 3 dual chambers.

### Example 9: Scaling the Bioreactor System into a Microplate-Based Format

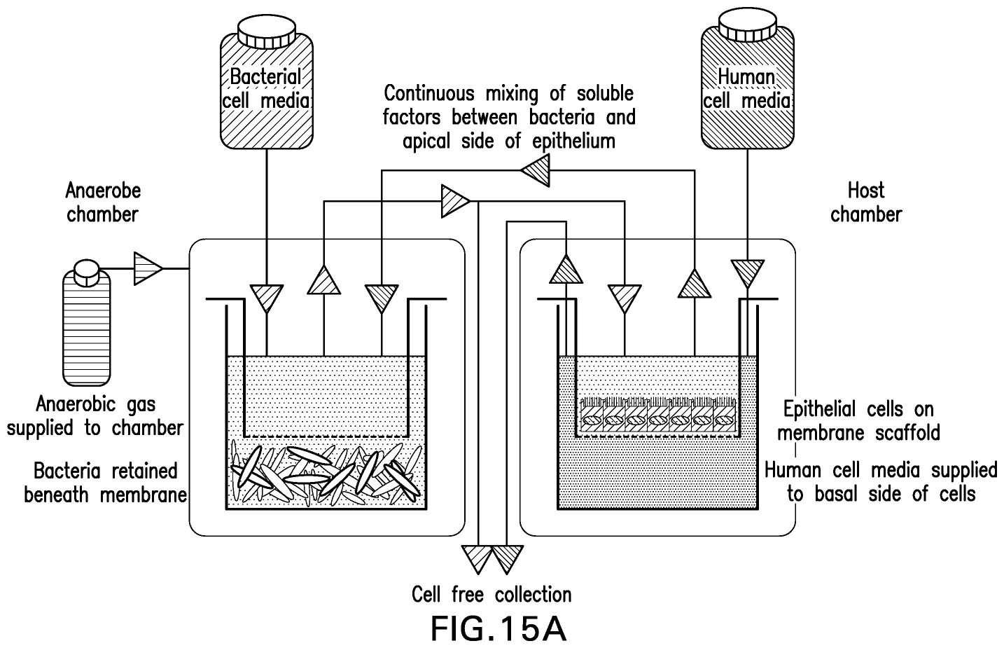

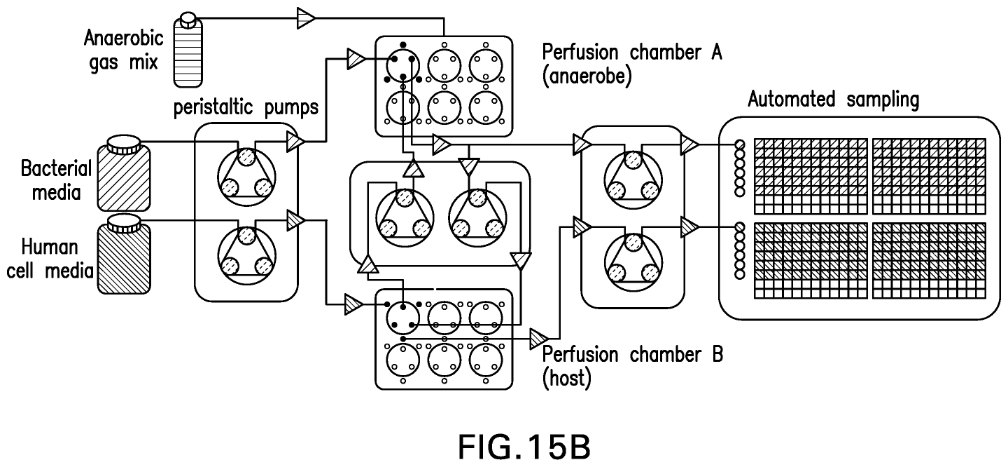

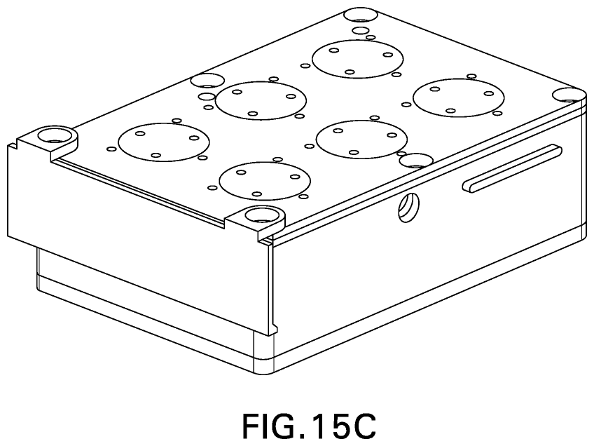

FIG. 15A demonstrates how the co-culture bioreactor system can be scaled into a microplate-based format. Transwell® inserts can be used as a permeable membrane to separate a chamber. The chamber is made up of a well in the microplate. The different environments to accommodate growth of different species are made in a microplate. The entire microplate is confined in a containment housing. The contents of a chamber in a microplate in a containment housing can be exchanged with the contents of a chamber in a microplate in an adjacent containment housing. FIG. 15A is a schematic diagram of the host-microbe co-culture perfusion bioreactor scaled into a microplate-based format. This figure is used for illustrative purposes to represent the cells in each chamber, and as an example, a bacterial chamber and an epithelial chamber comprising human cells. Chamber 1 is a bioreactor comprising a bacterial culture comprising a membrane, wherein the membrane is a Transwell® permeable membrane insert. Chamber 2 is a bioreactor comprising an epithelial culture comprising a membrane, wherein the membrane is a Transwell® permeable membrane insert. Human cell media is supplied directly to the basal side of the cells. Arrowed lines on schematic are representative of the fluidic tubing (and flow direction) on the apparatus and are not drawn to scale.

FIG. 15B shows an example of a configuration where two separate containment housings are positioned around peristaltic pumps that exchange the contents of the chambers and perfuse media.

FIG. 15C is a rendered image of an example of the custom containment housing used for the microplate (6-well plate) version. The containment housing serves as insulation for the internal environment from the external environment and holds the fluidic tubing in the correct locations to access both the top of the Transwell® permeable membrane insert and beneath the insert. It is made of four (4) layers. The first two layers at the top have a template for the microplate format being used, and also sandwich a rubber mat that serves as a septum. The septum also grips the metal tubing in the correct spot. The third layer is made up of the wall that holds the top two layers above the microplate. The third layer also has ports for gas entry/exit and notches for gripping. The bottom layer forms the foundation for the system. Also shown in FIG. 15C is a removable panel to attach heat pads for temperature control. FIG. 15B is a schematic diagram of a potential configuration of the host-microbe co-culture perfusion bioreactor when scaled into a microplate-based format. Peristaltic pumps can be used to transport media throughout the system. This diagram represents a 6 well plate as each perfusion chamber in a containment housing.

The chamber could also be made up of the microplate consisting of multiple wells. The wells in this environment would be exposed to the same gas and temperature environment and as a whole could represent a chamber.

In another example, a chamber could comprise of one well on the microplate, and a second chamber could comprise one well on the same microplate, where the two chambers are exposed to the same environment conditions. In this scenario, the contents of a chamber in a microplate in a containment housing can be exchanged with the contents of a chamber in the same microplate in the same containment housing.

### Example 10: Model Dye Injected in One Chamber can be Detected Throughout the Microplate-Based Format Bioreactor System Over Time

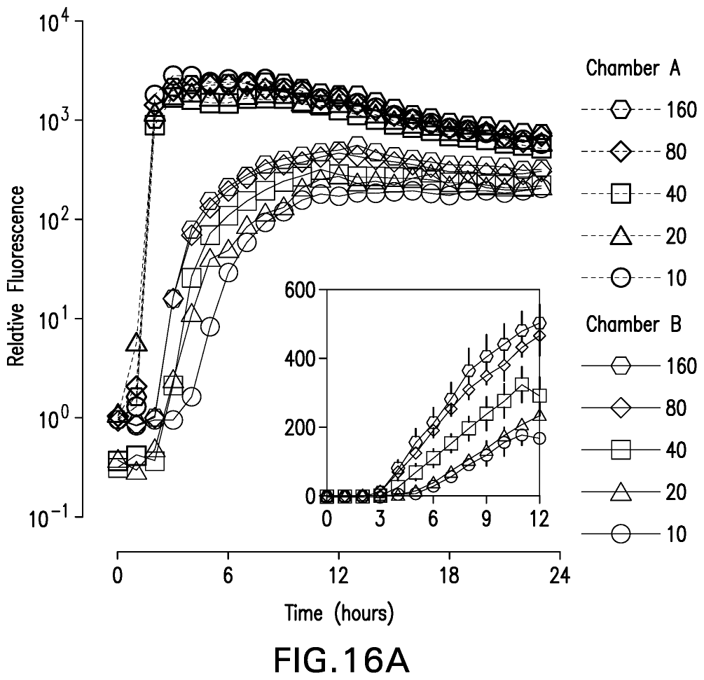

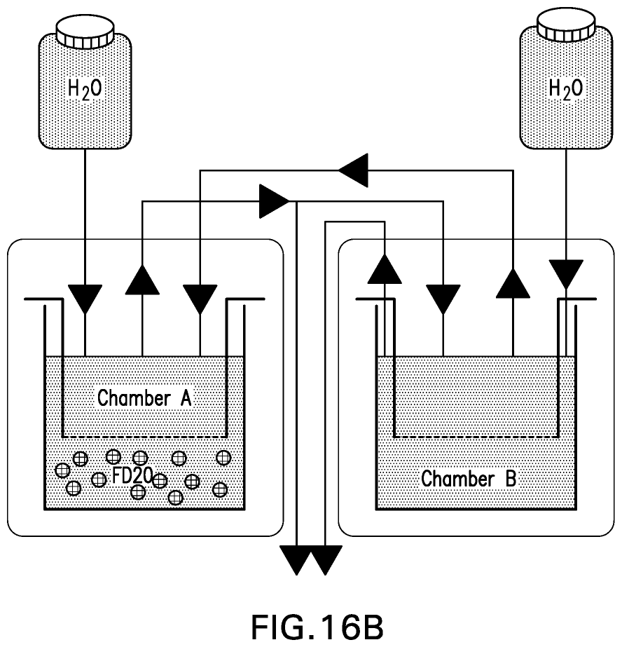

### FIGS. 16A-B

Injection of model dye was tracked throughout the microplate-based format bioreactor system in a time-dependent manner. Transwell®s (Corning 3450) were placed in 6-well plate, which was placed in the containment housing. In chamber 1, metal tubing height was adjusted to allow 3 ml of fluid beneath the Transwell® membrane and 1.5 ml of fluid above the Transwell®. In chamber 2, metal tubing height was adjusted to allow 5 ml of fluid beneath the Transwell® membrane and 2 ml of fluid above the Transwell®. Peristaltic pumps were turned on so that water was being added into and out of the chamber at 0.005 ml/min. Two fluidic pumps were used to circulate the water from above the Transwell membrane in chamber 1 to beneath the Transwell membrane in chamber 2 (and the reverse direction from beneath the Transwell membrane in chamber 2 to above the Transwell membrane in chamber 1) at varying flow rates ranging from 0.01 ml/min to 0.16 ml/min. Samples coming from chamber output pumps were collected at predetermined time intervals over the course of 24 h. At the start of the experiment, 1 mg/ml FD20 was added beneath the membrane in chamber 1 as represented in FIG. 16B. Output samples were loaded on flat, black, opaque bottom 96-well plates (Costar) and fluorescence was measured using a PHERAstar® plate reader (480/520). As shown in FIG. 16A, fluorescence measurement was used to measure FD20 in different locations of the microplate-based format bioreactor system over time. The location directly proximal to initial input of FD20 (i.e. chamber 1, represented by Chamber A in FIG. 16A, above the Transwell® membrane) shows higher fluorescence in comparison to the location furthest from FD20 input (i.e. chamber 2, represented by Chamber B in FIG. 16A, beneath the Transwell® membrane). FIG. 16A is a line graph showing FD20 concentration in chamber 1 and chamber 2, over 24 hours at varying flowrates (10 μl/min-160 μl/min). Chamber 1 is represented by the top data set comprising various flowrates and chamber 2 is represented by the bottom data set also comprising various flowrates and is shown in detail in the box. FIG. 16A corresponds with Example 10. Increases in FD20 concentration in chamber 1 were seen over time due to diffusion through membrane until a peak is reached, along with a slow decay due to perfusion. It appears from FIG. 16A, in Chamber B, Gradual increases in FD20 concentration in chamber 2 were seen. It appears from FIG. 16A, in Chamber B, the slower flowrates had a slightly lower and delayed relative fluorescence curve. FIG. 16B is a schematic diagram representing a two-chamber system as described in Example 10. The dual chamber system comprises H₂O supplied to the outside of the membrane in chamber 1 and the outside of the membrane in chamber 2. The two arrows pointing downwards in the middle of the two chambers are connected to the automated sample collection (fraction collector). Arrowed lines on schematic are representative of the fluidic tubing (and flow direction) on the apparatus and are not drawn to scale.

### Example 11: Establishing Anoxic Conditions in an Individual Chamber while Maintaining Aerobic Conditions in Separate Chamber in Microplate-Based Format Bioreactor System

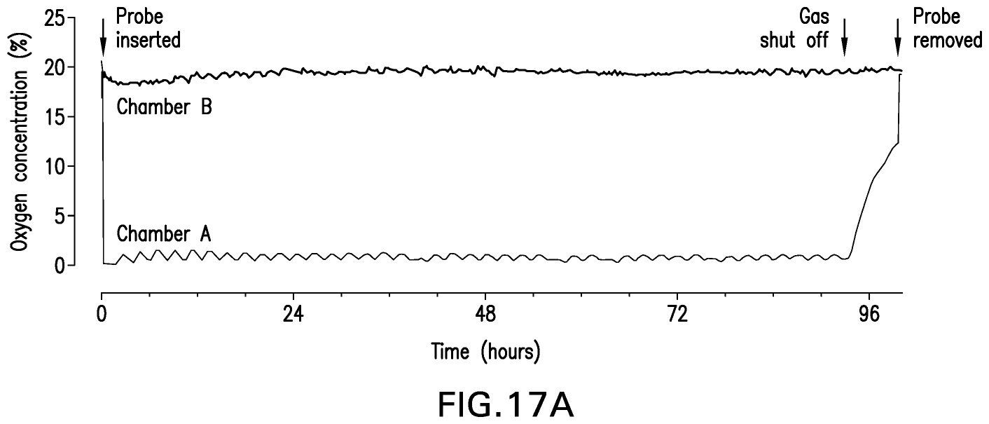

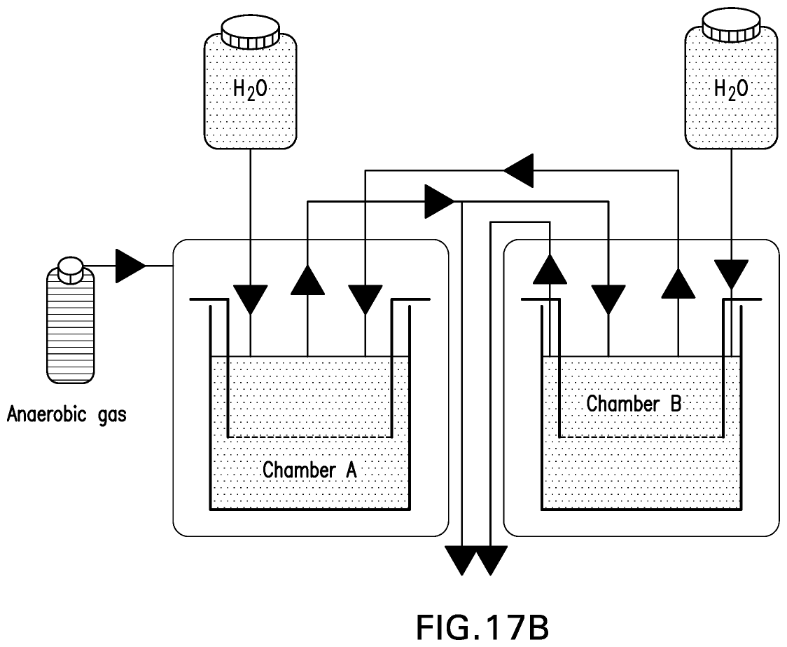

FIG. 17B is a schematic diagram representing a two-chamber system as described in Example 11. The dual chamber system comprises H₂O supplied to the outside of the membrane in chamber A and the outside of the membrane in chamber B. Anaerobic gas is supplied to the containment housing that holds Chamber A. Arrowed lines on schematic are representative of the fluidic tubing (and flow direction) on the apparatus and are not drawn to scale. The bioreactor system shown in FIG. 17B comprises two chambers. Two 0.5-liter bottles of water were used to supply the bioreactor systems. A 6-well microplate with Transwell® was pre-filled with water in an anaerobic chamber and placed in a containment housing (chamber A). In chamber A, metal tubing height was adjusted to allow 3 ml of fluid beneath the Transwell® membrane and 1.5 ml of fluid above the Transwell®. In chamber B, metal tubing height was adjusted to allow 5 ml of fluid beneath the Transwell® membrane and 2 ml of fluid above the Transwell®, and the microplate was pre-filled accordingly. The containment housing setup was removed from the chamber and tubing from an anerobic gas mix (Middlesex Gases & Technologies) was quickly connected to the chamber A containment housing and turned on. As shown in FIG. 17B, the bioreactor system was assembled with two chambers, each with a membrane attached inside. Two oxygen microsensors (PreSens) were inserted into two locations within the bioreactor system: (i) chamber A, below the membrane; and (ii) chamber B, below the membrane. All other fluidics pumps and tubing (inputs, outputs, and mixing) were connected. Anaerobic gas mix was released into system, oxygen concentration measurements were started, and fluidic pumps were turned on so that water was being added and removed at 0.005 ml/min in each chamber. Peristaltic pumps were used to circulate the water from above the membrane in chamber A to above the membrane in chamber B (and reverse direction) at 0.02 ml/min. Oxygen concentration measurements were recorded for approximately 4 days and are shown in FIG. 17A. FIG. 17A is a line graph showing oxygen concentration measurements for approximately 4 days in 2 locations within the bioreactor system (6-well microplate format): (i) Chamber A, beneath the membrane; (ii) Chamber B, above the membrane. FIG. 17A corresponds with Example 11.

Oxygen levels in chamber A dropped significantly upon placing probe in chamber A. Oxygen levels stayed consistently around 1% v/v for a period of nearly 4 days (chamber 1). Around 96 h, the sensors were removed as the bioreactor system was disassembled and sensors recorded typical atmospheric oxygen levels. The oxygen level inside the membrane in chamber B (where mammalian cells might be located in some embodiments) remained between 15-20% throughout the duration of the experiment, demonstrating an oxygenated environment.

### Example 12: Growth of Anaerobic Bacteria in Co-Culture with Caco-2 Cells (Human Epithelial) in Microplate-Based Format Bioreactor System

Example 12 comprised a series of bioreactor systems.

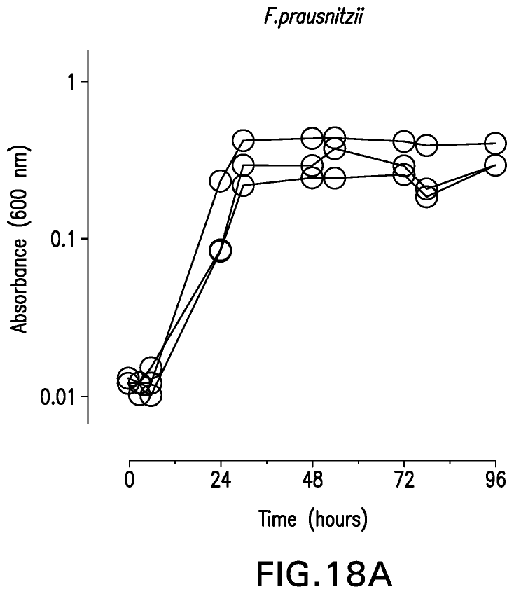

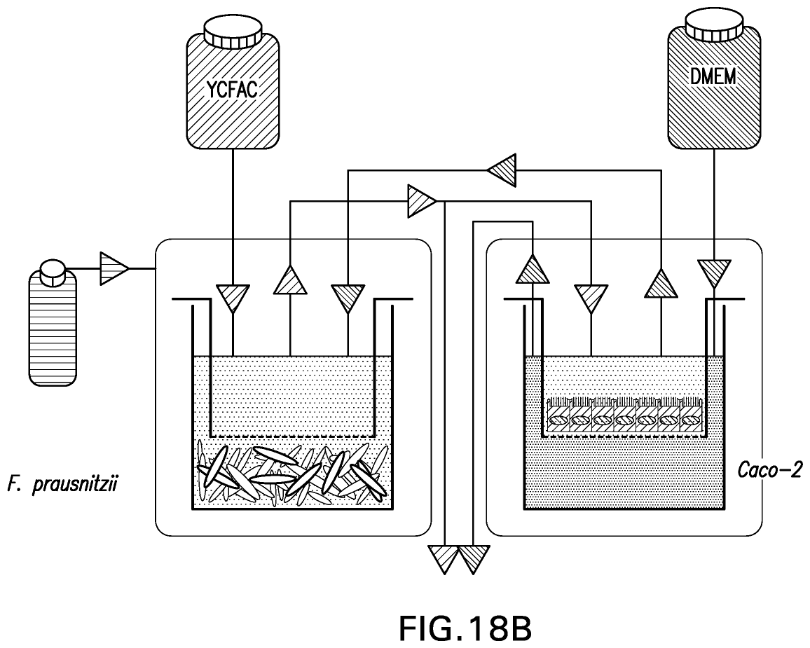

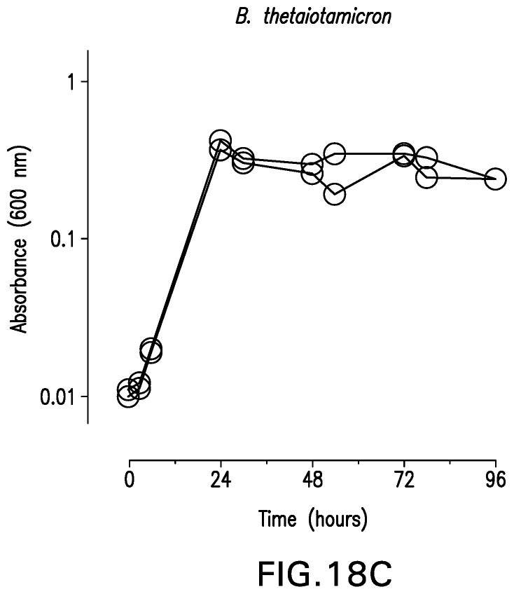

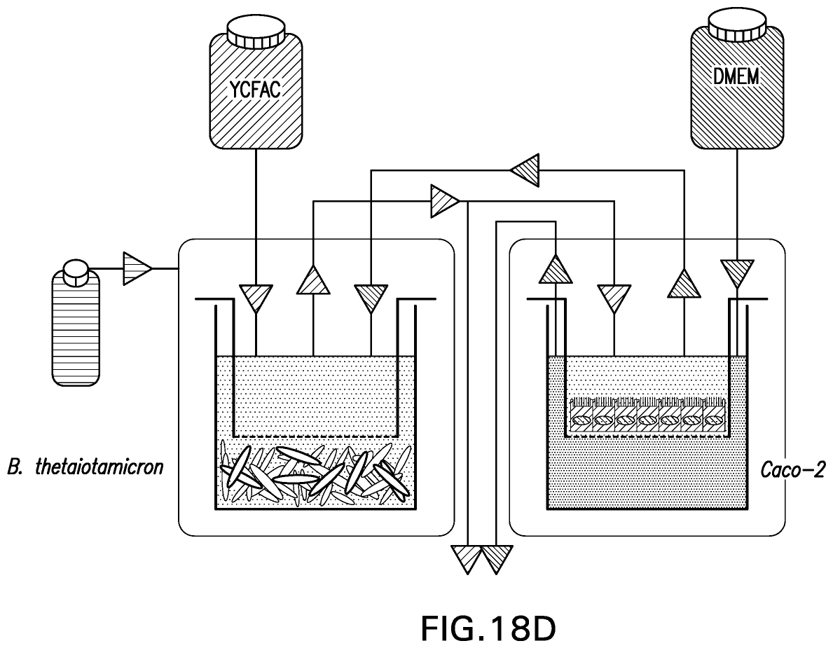

FIG. 18B is a schematic diagram representing the dual chamber the 6-well microplate format bioreactor system as described in Example 12. The dual-chamber bioreactor comprises YCFAC broth supplied to chamber 1 and DMEM supplied to chamber 2. Additionally, the bioreactor comprises an anaerobic gas mixture connected to the containment housing Chamber 1. Chamber 1 comprises bacterial cells (F. prausnitzii). Chamber 2 comprises epithelial cells (Caco-2 cells) with a tissue-culture treated membrane. Arrowed lines on schematic are representative of the fluidic tubing (and flow direction) on the apparatus and are not drawn to scale.

FIG. 18D is a schematic diagram representing the dual chamber the 6-well microplate format bioreactor system as described in Example 12. The dual-chamber bioreactor comprises YCFAC broth supplied to chamber 1 and DMEM supplied to chamber 2. Additionally, the bioreactor comprises an anaerobic gas mixture connected to the containment housing Chamber 1. Chamber 1 comprises bacterial cells (B. thetaiotaomicron). Chamber 2 comprises epithelial cells (Caco-2 cells) with a tissue-culture treated membrane. Arrowed lines on schematic are representative of the fluidic tubing (and flow direction) on the apparatus and are not drawn to scale.

Caco-2 cells grow in a monolayer on a membrane and display epithelial barrier function. Caco-2 cells were seeded on Transwell® membranes (Corning 3450) for 3 days, seeded at 500,000 cells per Transwell®. Three days after cells were seeded, media was removed by aspiration from all wells (above and below membrane) and used for an experiment in the bioreactor system.

Before initiating the separate experiments, Bacteroides thetaiotaomicron VPI 5482 (ATCC 29148) and Faecalibacterium prausnitzii A2-165 (DSMZ17677) were individually streaked onto YCFAC agar plates (Anaerobe Systems), and the plates were incubated at 37° C. in an anaerobic chamber. 24 h prior to the start of the experiment, individual colonies were picked and used to inoculate 5 ml of YCFAC broth (Anaerobe Systems), which were incubated at 37° C. in an anaerobic chamber.

The containment housing was sterilized by autoclaving. A 6-well microplate with Transwell® was pre-filled with YCFAC in an anaerobic chamber and placed in a containment housing (chamber A). The overnight cultures of B. thetaiotaomicron and F. prausnitzii (45 μl) were individually transferred to beneath the membrane in chamber 1 via a micropipet. In chamber 1, metal tubing height was adjusted to allow 3 ml of fluid beneath the Transwell® membrane and 1.5 ml of fluid above the Transwell®. The containment housing setup was removed from the chamber and tubing from an anerobic gas mix (Middlesex Gases & Technologies) was quickly connected to the chamber 1 containment housing and turned on to maintain a positive pressure with anaerobic gas. In chamber 2, metal tubing height was adjusted to allow 5 ml of fluid beneath the Transwell® membrane containing Caco-2 cell monolayer and 2 ml of fluid above the Transwell® containing Caco-2 cell cell monolayer, and the microplate was pre-filled with DMEM supplemented with 10% FBS (v/v) accordingly. As shown in FIG. 18B and FIG. 18D, YCFAC broth comprised the media bottle supplying chamber 1. DMEM with 10% FBS (v/v) comprised the media bottle supplying chamber 2. YCFAC media was pumped into chamber 1 above the membrane and DMEM with 10% FBS was pumped into chamber 2 beneath the membrane with Caco-2 cell monolayer. Peristaltic pumps were initiated at flow rates of 0.005 ml/min into and out of each chamber and 0.02 ml/min back-and-forth between above the membrane in chamber 1 and beneath the membrane with Caco-2 cell monolayer in chamber 2. The temperature was set to 37° C.

At various timepoints samples were removed from beneath the membrane in chamber 1 using a needled syringe, 100 μl was aliquoted onto a clear 96-well plate, and absorbance at 600 nm was measured using a PHERAstar FSX multi-mode microplate reader.

As shown in FIG. 18A (F. prausnitzii) and FIG. 18C (B. thetaiotaomicron), both bacterial species were able to grow in the bioreactor system in YCFAC media when co-cultured with Caco-2 cells and DMEM with 10% FBS. FIG. 18A is a line graph showing the results of Example 12, where F. prausnitzii is able to grow in the 6-well microplate format bioreactor system in anaerobic YCFAC media when co-cultured with Caco-2 cells and DMEM with 10% Fetal Bovine Serum (FBS). FIG. 18C is a line graph showing the results of Example 12, where B. thetaiotaomicron is able to grow in the 6-well microplate format bioreactor system in anaerobic YCFAC media when co-cultured with Caco-2 cells and DMEM with 10% Fetal Bovine Serum (FBS).

1. A perfusion bioreactor system comprising:
a. a first chamber comprising:
i. an inner membrane defining an inner volume, and
ii. an outer wall surrounding the inner membrane and defining an outer volume surrounding the inner membrane;
b. a second chamber comprising:
i. an optional inner membrane defining an inner volume, and
ii. an outer wall surrounding the optional inner membrane, wherein if the inner membrane is present, then the outer wall defines an outer volume surrounding the inner membrane, and if the inner membrane is not present, then the outer wall defines an outer volume comprising all the contents in the chamber;
c. a hollow conduit connecting the first and second chambers via an orifice in each of the first and second chambers, wherein the hollow conduit allows fluid communication between the first and second chamber; and
d. a first pump in mechanical contact with or in fluid communication with the hollow conduit, the first pump to move fluid between the first chamber and the second chamber,
wherein the inner membrane of each chamber houses cells in the inner volume, and wherein each chamber comprises a suitable medium for culturing the cells.

2. (canceled)

3. The bioreactor system of claim 1, wherein the first chamber comprises:
(a) at least one sampling orifice in fluid communication with the inner volume that allows removal of fluid from the inner volume,
(b) at least one sampling orifice in fluid communication with the outer volume that allows removal of fluid from the outer volume, and
wherein the second chamber comprises:
(a) at least one sampling orifice in fluid communication with the inner volume that allows removal of fluid from the inner volume,
(b) at least one sampling orifice in fluid communication with the outer volume that allows removal of fluid from the outer volume.

4. (canceled)

5. (canceled)

6. (canceled)

7. The bioreactor system of claim 1, wherein fluid removed from the outer or inner volume of the first chamber is mixed in the second chamber via an orifice.

8. The bioreactor system of claim 1, wherein fluid removed from the outer or inner volume of the second chamber is mixed in the first chamber via an orifice.

9. The bioreactor system of claim 1, wherein the inner or outer volume of the first chamber is mixed to expose the second chamber to fluid containing secreted products from the cells cultured in the first chamber, and wherein the inner or outer volume of the second chamber is mixed to expose the first chamber to fluid containing secreted products from the cells cultured in the second chamber.

10. The bioreactor system of claim 1, wherein at least one chamber has an aerobic environment.

11. The bioreactor system of claim 1, wherein at least one chamber has an anaerobic environment.

12. (canceled)

13. (canceled)

14. The bioreactor system of claim 1, wherein the first chamber has an aerobic environment and the second chamber has an anaerobic environment.

15. The bioreactor system of claim 10, wherein each aerobic chamber houses mammalian cells, human cells, microbial cells or epithelial cells.

16. (canceled)

17. (canceled)

18. The bioreactor system of claim 11, wherein the anaerobic environment chamber houses bacterial cells.

19. The bioreactor system of claim 18, wherein the bacterial environment comprises a mucin-coated membrane.

20. The bioreactor system of claim 1, wherein the chamber comprises human epithelial cells within a matrix-coated membrane scaffold.

21. The bioreactor system of claim 1 wherein the first chamber comprises human epithelial cells growing on a porous membrane scaffold and the cells in the second chamber are bacterial cells and the optional membrane of the second chamber is a mucin-coated membrane.

22. The bioreactor system of claim 1, wherein one chamber has microbial cells and the other chamber has epithelial cells.

23. The bioreactor system of claim 22 wherein the microbial cells comprise bacterial cells.

24. (canceled)

25. The bioreactor system of claim 1, wherein the cells can sense and respond to secreted signals.

26. (canceled)

27. A method of emulating the gastrointestinal tract, comprising the bioreactor system of claim 3, wherein the bioreactor system replenishes cell culture media during the experiment allowing for multiple samples to be collected enabling various analytical techniques and results.

28. (canceled)

29. (canceled)

30. The bioreactor system of claim 3, wherein the sample volumes collected from multiple chambers in parallel enable various analytical techniques and results.

31. The bioreactor system of claim 3, wherein the bioreactor system enables sample volumes up to about 100 ml to be collected on multiple occasions throughout a co-culture process.

32. A method of emulating the gastrointestinal tract, comprising culturing cells in a suitable medium in a bioreactor system, wherein the bioreactor system comprises a plurality of chambers, wherein a first chamber comprises an inner membrane defining an inner volume and an outer wall surrounding the inner membrane and defining an outer volume surrounding the inner membrane, and a second chamber comprising an optional inner membrane defining an inner volume, and an outer wall surrounding the optional inner membrane, wherein if the inner membrane is present, then the outer wall defines an outer volume surrounding the inner membrane, and if the inner membrane is not present, then the outer wall defines an outer volume comprising all the contents in the chamber, wherein each chamber houses cells cultured in a suitable medium and environmental conditions, and wherein the chambers have one or more orifices through which a hollow conduit connects the chambers and allows them to be in fluid communication, wherein the contents from the chambers are mixed and circulated with the other chamber(s),
wherein the chambers each comprise an inner membrane and wherein the membrane divides each chamber into two sections, the outer volume and inner volume,
wherein the mixing exposes the cell culture of each chamber to the secreted products of the other chamber(s) enable communication across the cells grown in separate environments, wherein the communication comprises the cells responding to the secreted signals upon co-culture, and
wherein the contents from outer volume in one chamber is mixed with inner volume of the second chamber, and wherein the contents in the inner volume of the second chamber is mixed with the contents in the outer volume in the first chamber.

33. The method of claim 32, comprising culturing epithelial cells in one chamber of a two chamber bioreactor system and culturing microbial cells in a second chamber of the bioreactor system, wherein both cell culture contents comprising secreted product from the cell cultures are mixed and circulated between the two chambers.

34. (canceled)

35. (canceled)

36. The method of claim 32, wherein the cells bioluminescence in response to the secreted signals from a co-culture.

37. (canceled)

38. (canceled)

39. (canceled)

40. The method of claim 32, wherein the bioreactor system is used at room temperature, 30 degrees Celsius, or 37 degrees Celsius.

41. (canceled)

42. (canceled)

43. The method of claim 32, wherein the bioreactor system is used at ambient environment gas conditions.

44. The method of claim 32, wherein the bioreactor system is used at environment gas conditions between 0.1% O₂ v/v to about 21% O₂ v/v.

45. (canceled)

46. (canceled)

47. The bioreactor system of claim 1, wherein the bioreactor system comprises more than one well on a microplate within the first chamber and comprises more than one well on the microplate within the second chamber.

48. (canceled)

49. The method of claim 27, wherein the sample volumes collected from multiple chambers in parallel enable various analytical techniques and results.
